(19) United States
(12) Patent Application Publication
(10) Pub. No.: US 2004/0005601 A1
(43) Pub. Date: Jan. 8, 2004

(54) Methods for targeting quadruplex DNA

(76) Inventors: Adam Siddiqui-Jain, San Diego, CA (US); Laurence H. Hurley, Tucson, AZ (US); Thomas J. Farrell, Del Mar, CA (US); Cory L. Grand, Tucson, AZ (US); David J. Bearss, Modesto, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/407,449

(22) Filed: Apr. 4, 2003

Related U.S. Application Data
(60) Provisional application No. 60/456,637, filed on Mar. 20, 2003.
(60) Provisional application No. 60/404,966, filed on Aug. 20, 2002.
(60) Provisional application No. 60/370,358, filed on Apr. 5, 2002.

(51) Int. Cl. C12Q 1/68

(52) U.S. Cl. 435/6

(57) ABSTRACT

Among the different intrastrand quadruplex structures that can arise from duplex DNA, it has been discovered that a chair conformation is biologically significant. Also, it has been determined that certain mutations in quadruplex forming nucleotide sequences destabilize quadruplex structure and are associated with cancer. Thus, provided herein are quadruplex-destabilized nucleic acid acids, cancer diagnostics and prognostics, methods for using the cancer diagnostics and prognostics to prevent and/or treat cancer, nucleic acid therapeutics that target quadruplex-destabilized nucleotide sequences and methods, methods for identifying compounds that modulate the biological activity of a native quadruplex DNA in a chair conformation, and methods for modulating the biological activity of a native quadruplex DNA with a compound identified by the methods described herein.

### RELATED PATENT APPLICATIONS

This patent application claims the benefit of provisional patent application 60/404,966 filed Aug. 20, 2002 and provisional patent application 60/370,358 filed Apr. 5, 2002, having attorney docket number 532233000400 and 532233000401, respectively. Each of these provisional patent applications is entitled “Methods of regulating transcription by targeting quadruplex DNA” and names Adam Siddiqui-Jain et al. as inventors. This patent application also claims the benefit of a provisional application filed on Mar. 20, 2003, having attorney docket number 532233000402, entitled “Methods for targeting quadruplex DNA,” and names Adam Siddiqui-Jain et al. as inventors (application number not available at the time this application is filed). Each of these three provisional patent applications is hereby incorporated herein by reference in its entirety, including all drawings and cited documents.

### STATEMENT OF GOVERNMENT SUPPORT

This invention was made in part with government support under Grant Nos. CA67760 and CA88310 awarded by the National Institutes of Health. The government has certain rights in this invention.

### FIELD OF THE INVENTION

The invention relates to DNA sequences capable of forming a particular class of secondary structure referred to as a quadruplex.

### BACKGROUND

Developments in molecular biology have led to an understanding of how certain therapeutic compounds interact with molecular components and lead to a modified physiological condition. Specificity of therapeutic compounds for their targets is derived in part from complementary structural elements between the target molecule and the therapeutic compound. A greater variety of target structural elements leads to the possibility unique and specific target/compound interactions. As a result, researchers have focused heavily on polypeptides as targets for the design of specific therapeutic molecules as polypeptides are structurally diverse.

In addition to therapeutic compounds that target polypeptides, researchers also have identified compounds which target DNA. Some of these compounds are effective anticancer agents and have led to significant increases in the survival of cancer patients. Unfortunately, however, these DNA targeting compounds do not specifically act on cancer cells and are therefore extremely toxic. One reason why DNA targeting compounds may be unspecific is DNA requires the uniformity of Watson-Crick duplex structure for compactly storing information within the human genome. This uniformity of DNA structure does not lead to a structurally diverse population of DNA molecules where each of the molecules can be specifically targeted.

Nevertheless, there are some exceptions to this structural uniformity, as certain DNA sequences can form unique secondary structures. For example, intermittent runs of guanines can form quadruplex structures, and complementary runs of cytosines can form i-motif structures. Formation of quadruplex and i-motif structures occurs when a particular region of duplex DNA transitions from Watson-Crick base pairing to single-stranded structures. While quadruplex DNA structures readily form under physiological conditions, formation of i-motif structures require acidic conditions, which makes their physiological relevance less likely, but still possible.

Quadruplex structures can vary in several different ways, including strand stoichiometry and strand orientation. For example, interstrand quadruplex structures can form when four strands form a parallel quadruplex structure or two strands form a hairpin quadruplex structure. Intrastrand quadruplex structures may form when a single strand forms a basket quadruplex conformation or a chair quadruplex conformation. Variation among these forms can arise in the loop structures of hairpin, basket, and chair quadruplexes, where the loops may consist of any of the four bases in loop sizes of two- to six-base pairs (e.g. see FIG. 1).

Researchers have determined that quadruplex structures form in telomere DNA and have targeted these structures for the design of anticancer compounds. It was thought that sequestering the single-stranded DNA primer in a quadruplex structure would inhibit telomerase by eliminating the substrate required for reverse transcriptase activity of the telomerase. See e.g. Sun et al, J. Med. Chem. 40: 2113-2116 (1997). Inhibiting telomerase was thought to result in shortened telomere length, which may result in cell death, and cancer cells with one abnormally short telomere presumably would more sensitive than non-cancerous cells to these telomerase inhibitors.

Quadruplex-forming sequences have also been identified in transcriptional regulatory regions of oncogenes, including CMYC, CMYB, CFOS, and CABL. Regulatory regions of these oncogenes include DNA sequences which can form single-stranded regions hypersensitive to nucleases. In the CMYC promoter, the regions which can form single-stranded structures bind transcription factors, such as cellular nucleic acid-binding protein (CNBP) and heterogeneous nuclear ribonucleoprotein (hnRNP), which are presumably required for transcriptional activation. Also, the interconversion between paranemic forms (e.g., unwound and non-B forms) and single stranded forms of regions in the CMYC promoter is proposed to require NM23-H2 as an accessory factor (see, e.g., Postel et al., J. Bioenerg. Biomembr. 32: 277-284 (2000)). As these elements may potentially form G-quadruplex and i-motif structures, it is possible that the secondary DNA structures inactivate transcription, and their conversion to the duplex region is required for transcriptional activation. Researchers studying an insulin-linked polymorphic region (ILPR) in the insulin gene postulated that these regions regulate insulin function in insulin-dependent diabetes mellitus via quadruplex structures (Lew et al., Proc. Natl. Acad. Sci. U.S.A. 97: 12508-12512 (2000)).

Some research has been devoted to determining whether certain compounds bind quadruplex structures. It was shown that the cationic porphyrin TMPyP4 was able to bind to G-quadruplex structures, and the analog TMPyP2 bound with less affinity because of restricted rotation around the meso bond, which is required for insertion into and stacking external to the G-tetrad structure (Han et al., J. Am. Chem. Soc. 121: 3561-3570 (1999)). Consequently, biochemical effects, such as inhibition of telomerase and inhibition of helicase-mediated unwinding of G-quadruplex structures, have been associated with only TMPyP4. Corresponding biological effects, such as production of anaphase bridges and in vivo antitumor activity, also have been associated only with TMPyP4. Also, it was shown that TMPyP4, but not TMPyP2, lowered CMYC transcription and protein expression, as well as downstream genes, such as hTERT, ODC, and CDC25A. These studies, however, do not clearly show which quadruplex structure is bound by the compounds.

Some research has lead to structural determinations of quadruplex DNA. X-ray and crystallographic studies have been derived for the thrombin binding aptamer (TBA) and an HIV-integrase binding oligonucleotide (typified by T30695) (Schultze et al., J. Mol. Biol. 235: 1532-1547 (1994); Kelly et al., J. Mol. Biol. 256: 417-422 (1996); Jing et al., J. Biomol. Struct. Dyn. 15: 573-585 (1997); Jing et al., J. Biol. Chem. 273: 34992-34999 (1998)). Both aptamers consist of two stacked G-tetrads and two lateral two-base loops. Whereas the bridging loop of TBA contains 3 bases, for T30695 there are only two. Both structures contain a TGTG motif in the lateral loop region, which for T30695 has been proposed to form an ordered tetrad arrangement. In an effort to determine which quadruplex structure might be relevant for regulating transcription, researchers studying the CMYC regulatory region in vitro postulated that the quadruplex structure was stabilized by potassium ions in a basket conformation (Simonsson et al., Nucleic Acids Res. 26: 1167-1172 (1998)).

Thus, while some information is available concerning quadruplex DNA, a need exists for elucidating which conformations are biologically significant. Such a determination would provide valid targets for the discovery of therapeutic compounds that can interact with quadruplexes and modulate their biological function.

### SUMMARY

Certain regulatory regions in duplex DNA can transition into single stranded structures, including intrastrand quadruplex structures. These regulatory regions can form at least two different intramolecular quadruplex conformations. One is a basket conformation, which forms slowly into a stable structure and is therefore thermodynamically favored. The second is a chair conformation, which forms more rapidly than the basket conformation, and is therefore kinetically favored. Of these two structures, it has been discovered that the chair conformation rather than the basket conformation is biologically significant. Identifying this biologically significant conformation of G-quadruplexes paves the way for identifying compounds that specifically interact with a quadruplex DNA structure in vivo.

Thus, featured herein is a method for identifying a compound that modulates the biological activity of a native quadruplex DNA, which comprises contacting a test quadruplex DNA in the chair conformation with a candidate compound, and determining the presence or absence of an interaction between the candidate compound and the test quadruplex DNA. One embodiment is a method for identifying a compound that binds a native quadruplex DNA, which comprises contacting a test quadruplex DNA in a chair conformation with a candidate compound, and determining the presence or absence of binding between the candidate and the test quadruplex DNA.

Also featured is a method for modulating the biological activity of a biologically significant native quadruplex DNA, which comprises contacting a system comprising the native quadruplex DNA with a compound which interacts with a quadruplex DNA in a chair conformation.

It also has been discovered that the DNA of certain subjects having cancer includes a mutation that destabilizes a quadruplex structure that regulates transcription. Thus, featured herein are prognostic methods for determining whether a subject is at risk of developing or having cancer (e.g. colorectal cancer) by detecting one or more quadruplex-destabilizing mutations in a DNA sample from the subject. In a related embodiment, featured herein is a method for identifying a subject at risk of developing or having cancer by detecting the presence or absence of a quadruplex-destabilizing mutation in a DNA sample of the subject, and if a quadruplex-destabilizing mutation is detected in the DNA sample from the subject, targeting cancer prevention and/or treatment regimens to the subject. In one embodiment, disclosed herein is an antisense nucleic acid cancer therapy that specifically targets DNA in subjects having a quadruplex-destabilizing mutation.

### BRIEF DESCRIPTION OF THE DRAWINGS

FIG. 1 depicts generalized quadruplex conformations.

FIGS. 2A-2C show results from structural studies which demonstrate that Pu27 adopts a chair conformation and a basket conformation in vitro.

FIGS. 3A-3D show the effect of mutations of Pu27 on transcriptional activity and quadruplex stability.

FIGS. 4A-4D depict photocleavage and stabilization of basket and chair quadruplexes formed from Pu27 as a result of interaction with TMPyP4 and TMPyP2.

FIG. 5 shows the effect of TMPyP2 and TMPyP4 on CMYC mRNA synthesis in two different Burkitt's lymphoma cell lines.

FIG. 6A depicts a generalized quadruplex structure for CMYC, RET, and PDGFA. FIG. 6B depicts a quadruplex structure for RET. FIG. 6C depicts quadruplex structures for PDGFA.

FIG. 7 illustrates a quadruplex-forming nucleotide sequence in the promoter region upstream of the VEGF oncogene.

FIGS. 8A and 8B depict a RET quadruplex-forming nucleotide sequence and TMPyP4-induced photocleavage sites, respectively.

### DETAILED DESCRIPTION

An analysis described herein identified intramolecular chair quadruplex DNA structures as biologically relevant oncogene regulators. Thus, isolated quadruplex-forming DNA is useful for screening molecules that interact with quadruplex structures to identify lead cancer treatments. Another analysis described herein linked the occurrence of cancer with genetic alterations in human genomic DNA. These genetic alterations occurred at polymorphic sites that destabilize the chair quadruplex structure. In accordance with these findings, provided herein are quadruplex-destabilized nucleic acids, methods for determining whether a subject is at risk of developing or having cancer, pharmacogenomic methods for targeting appropriate prevention or therapeutic regimens to subjects identifying as being at risk of developing or having cancer, methods for screening molecules that interact with stabilized and destabilized quadruplexes, and therapeutic methods for treating cancers.

### Quadruplex Nucleic Acids and Variants Thereof

Quadruplex structures can form in certain purine-rich strands of nucleic acids. In the context of a duplex nucleic acid, certain purine rich strands are capable of engaging in a slow equilibrium between a typical duplex helix structure and both unwound and non-B-form regions. These unwound and non-B forms can be referred to as “paranemic structures,” and some forms are associated with sensitivity to S1 nuclease digestion, which can be referred to as “nuclease hypersensitivity elements” or “NHEs.” A quadruplex is one type of paranemic structure and certain NHEs can adopt a quadruplex structure.

As used herein, the term “quadruplex nucleic acid” and “quadruplex forming nucleic acid” refers to a nucleic acid in which a quadruplex structure may form. The entire length of the nucleic acid may participate in the quadruplex structure or a portion of the nucleic acid length may form a quadruplex structure. The term “test nucleic acid” as used herein refers to a nucleic acid that may or may not be capable of forming a quadruplex structure. Quadruplex-forning nucleic acids described herein are capable of forming a chair structure, as described in greater detail hereafter.

Quadruplex nucleic acids and test nucleic acids may comprise or consist of DNA (e.g., genomic DNA (gDNA) and complementary DNA (cDNA)) or RNA (e.g., mRNA, tRNA, and rRNA). In embodiments where a quadruplex nucleic acid or test nucleic acid is a gDNA or cDNA fragment, the fragment is often 50 or fewer, 100 or fewer, or 200 or fewer base pairs in length, and is sometimes about 300, about 400, about 500, about 600, about 700, about 800, about 900, about 1000, about 1100, about 1200, about 1300, or about 1400 base pairs in length. Methods for generating gDNA and cDNA fragments are well known in the art (e.g., gDNA may be fragmented by shearing methods and cDNA fragment libraries are commercially available). In embodiments where the quadruplex nucleic acid or test nucleic acid is a synthetically prepared oligonucleotide, the oligonucleotides can be about 8 to about 80 nucleotides in length, often about 8 to about 50 nucleotides in length, and sometimes from about 10 to about 30 nucleotides in length. In other words, the oligonucleotide often is about 80 or fewer, about 70 or fewer, about 60 or fewer, or about 50 or fewer nucleotides in length, and sometimes is about 40 or fewer, about 35 or fewer, about 30 or fewer, about 25 or fewer, about 20 or fewer, or about 15 or fewer nucleotides in length. Synthetic oligonucleotides can be synthesized using standard methods and equipment, such as by using an ABI™3900 High Throughput DNA Synthesizer, which is available from Applied Biosystems (Foster City, Calif.).

Quadruplex nucleic acids and test nucleic acids may comprise or consist of analog or derivative nucleic acids, such as peptide nucleic acids (PNA) and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WIPO publications WO 00/56746 and WO 01/14398, and related publications. Methods for synthesizing oligonucleotides comprising such analogs or derivatives are disclosed, for example, in the patent publications cited above, in U.S. Pat. Nos. 5,614,622; 5,739,314; 5,955,599; 5,962,674; 6,117,992; in WO 00/75372; and in related publications.

Often, a quadruplex nucleic acid or a test nucleic acid includes a nucleotide sequence that is identical to a native nucleotide sequence present in genomic DNA. For example, a quadruplex nucleic acid or a test nucleic acid may comprise or consist of a nucleotide sequence or a portion of a nucleotide sequence set forth in Table 1. The nucleotide sequences in Table 1 originate from regions in genomic DNA that regulate transcription of the CMYC, PDGFA (platelet derived growth factor alpha), PDGFB/c-sis (platelet derived growth factor beta), CABL, RET, BCL2, Cyclin D1/BCL1, KRAS, HRAS, and VEGF (vascular endothelial growth factor). An example of a test quadruplex DNA is Pu27, which is a 27 base-pair nucleic acid identical to the nucleotide sequence located −142 to −115 base-pairs upstream from the P1 promoter of the CMYC NHE III₁ (see SEQ ID NO: 1 in Table 1).

TABLE 1

| | | |
|---|---|---|
| SEQ | | |
| | ID | |
| Sequence | NO | Origin |

| | | |
|---|---|---|
| TG₄AG₃TG₄AG₃TG4AAGG | 1 | CMYC |
| CTAGAG₅CG₅CG₅CG₅AG₄T | 2 | PDGFA |
| G₈ACGCG₃AGCTG₅AG₃CTTG₄CCAG₃CG₄CGCTTAG₅ | 3 | PDGFB/ |
| | | c-sis |
| AGGAAG₄AG₃CCG₆AGGTGGC | 4 | CABL |
| G₅(CG₄)₃ | 5 | RET |
| G₃AGGAAG₅CG₃AGTCG₄ | 6 | BCL2 |
| G₄ACGCG₃CG₅CG₆AG₃CG | 7 | Cyclin |
| | | D1/BCL1 |
| (G₃A)₃AGGA(G₃A)₄GC | 8 | KRAS |
| G₅(CG₄)₃ | 9 | HRAS |
| C₃G₄CG₃C₂G₅CG₄TC₃G₂C4₅CG₂AG | 10 | VEGF |

While quadruplex forming sequences typically are identified in regulatory regions upstream of a gene (e.g., a promoter or a 5′ untranslated region (UTR)), quadruplex forming sequences also may be identified within a 3′ UTR, an intron or exon of a gene, and telomeric DNA.

A quadruplex nucleic acid or a test nucleic acid utilized in the methods described herein often includes a nucleotide sequence that is substantially similar, but not identical, to a native nucleotide sequence in genomic DNA. A quadruplex nucleic acid or a test nucleic acid often utilized in a system is in the chair form. Examples of nucleotide sequences capable of forming a chair quadruplex conform to the motif (GₐX_{b})_{c}Gₐ, where G is guanine; X is guanine, cytosine, adenine, or thymine; a is an integer between 2 to 10; b is an integer between 1 to 6; and c is the integer 3. Sometimes a is an integer between 2 and 6 and b is an integer between 1 and 4, and often, b is the integer 2 or 3. A quadruplex nucleic acid or a test nucleic acid may include one or more flanking nucleotides on the 5′ and/or 3′ end of the nucleotide sequence that forms the quadruplex that are not part of the quadruplex structure.

Substantially similar quadruplex nucleic acids often are nearly identical to native quadruplex nucleotide sequences and sometimes include one or more quadruplex-destabilizing nucleotide substitutions. Such alterations, which are also referred to hereafter as “polymorphisms,” may result from an insert, deletion, or substitution of one or more nucleotides. Such substitutions often are a single nucleotide replacement of a guanine that participates in a G-tetrad, where one, two, three, or four of more of such guanines in the quadruplex nucleic acid are substituted. For example, in certain cancer cells, native quadruplex nucleic acids described previously (e.g., Table 1) sometimes include nucleotide substitutions of one or more guanines that participate in a G-tetrad, where the nucleotide substitution is from guanine to another nucleotide (e.g., adenine) and destabilizes the quadruplex structure. For example, a quadruplex-destabilized nucleic acid sometimes comprises part of or all of the nucleotide sequence TGGGGAGGGTGXGGAGGGTGGGG, TGGGGAGGGTGGGGAGXGTGGGG, or TGGGGAGGGTGXGGAGXGTGGGG, where G is guanine, A is adenine, C is cytosine, T is thymine, and X is any nucleotide except for guanine, sometimes adenine. Methods for identifying quadruplex nucleotide sequences having destabilizing guanine substitutions in different cancer tissues and cells are described hereafter.

A native quadruplex nucleic acid can be converted to a quadruplex-destabilized nucleic acid by substituting one or more guanines that participate in a G-tetrad with another nucleotide (e.g. adenine). Such substitutions can be introduced by standard recombinant molecular biology techniques known in the art. One, two, three, or four or more guanines can be substituted in any quadruplex-forming nucleotide sequence, including the nucleotide sequences set forth in Table 1, and as described in specific embodiments and examples hereafter. A quadruplex-destabilized nucleic acid often is about 80 or fewer, about 70 or fewer, about 60 or fewer, or about 50 or fewer nucleotides in length, and sometimes is about 40 or fewer, about 35 or fewer, about 30 or fewer, about 25 or fewer, about 20 or fewer, or about 15 or fewer nucleotides in length.

Quadruplex nucleic acids and test nucleic acids may be contacted in the system as single-stranded nucleic acids, double stranded nucleic acids, or other forms of nucleic acids (see, e.g., Ren & Chaires, Biochemistry 38: 16067-16075 (1999)). Double stranded nucleic acids may be presented in the system by a plasmid, as exemplified herein.

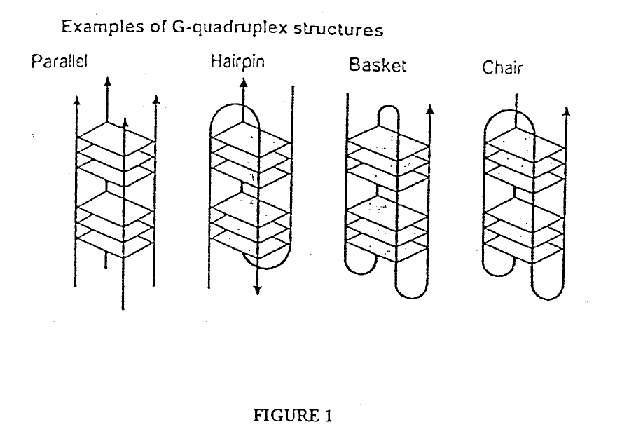

Quadruplex nucleic acids can exist in different conformations, which differ in strand stoichiometry and/or strand orientation. FIG. 1 illustrates examples of different interstrand and intrastrand quadruplex structures. The ability of guanine rich nucleic acids of adopting these structural conformations is due to the formation of guanine tetrads through Hoogsteen hydrogen bonds. Thus, one nucleic acid sequence can give rise to different quadruplex orientations, where the different conformations depend in part upon the nucleotide sequence of the quadruplex nucleic acid and conditions under which they form, such as the concentration of potassium ions present in the system and the time that the quadruplex is allowed to form.

Different quadruplex conformations can be identified separately from one another using standard procedures known in the art, and as described herein. Also, multiple conformations can be in equilibrium with one another, and can be in equilibrium with duplex nucleic acid if a complementary strand exists in the system. The equilibrium may be shifted to favor one conformation over another such that the favored conformation is present in a higher concentration or fraction over the other conformation or other conformations. The term “favor” or “stabilize” as used herein refers to one conformation being at a higher concentration or fraction relative to other conformations. The term “hinder” or “destabilize” as used herein refers to one conformation being at a lower concentration. One conformation may be favored over another conformation if it is present in the system at a fraction of 50% or greater, 75% or greater, or 80% or greater or 90% or greater with respect to another conformation (e.g., another quadruplex conformation, another paranemic conformation, or a duplex conformation). Conversely, one conformation may be hindered if it is present in the system at a fraction of 50% or less, 25% or less, or 20% or less and 10% or less, with respect to another conformation.

Equilibrium may be shifted to favor one quadruplex form over another form by methods described herein. For example, certain bases in quadruplex DNA may be mutated to hinder or destabilize the formation of a particular conformation. Typically, these mutations are located in tetrad regions of the quadruplex (regions in which four bases interact with one another in a planar orientation), as demonstrated by one mutation to Pu27 that favored the chair form and another that favored the basket form. Also, ion concentrations and the time with which quadruplex DNA is contacted with certain ions can favor one conformation over another. For example, potassium ions stabilize quadruplex structures, and higher concentrations of potassium ions and longer contact times of potassium ions with quadruplex DNA tend to favor the basket conformation over the chair conformation of Pu27. The chair conformation is favored with contact times of 5 minutes or less in solutions containing 100 mM potassium ions, and often contact times of 10 minutes or less, 20 minutes or less, 30 minutes or less, and 40 minutes or less. Potassium ion concentration and the counteranion can vary, and the skilled artisan can routinely determine which quadruplex conformation exists for a given set of conditions by utilizing the methods described herein. Furthermore, compounds that interact with quadruplex DNA may favor one form over the other and thereby stabilize one form, as is shown herein with TMPyP4, which stabilizes the chair conformation over the basket conformation of Pu27.

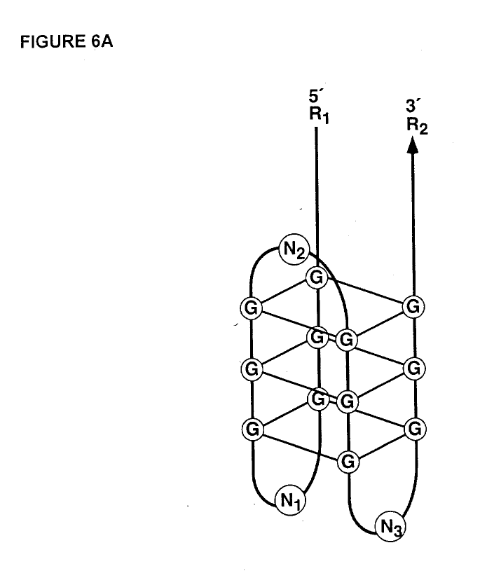

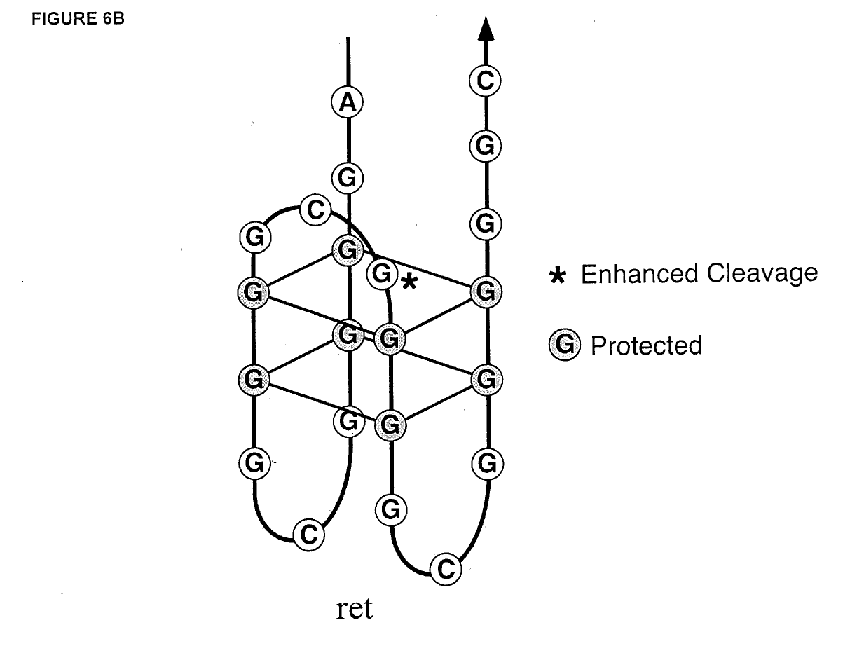

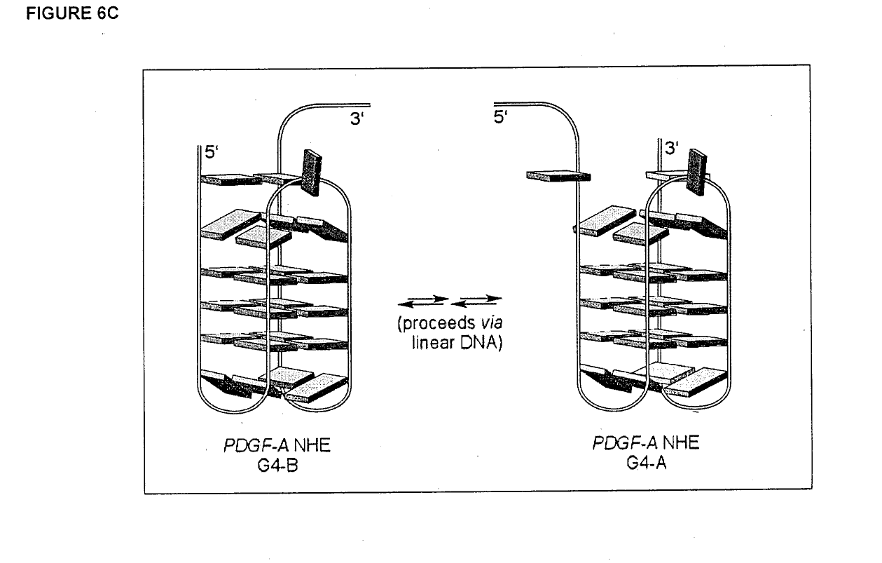

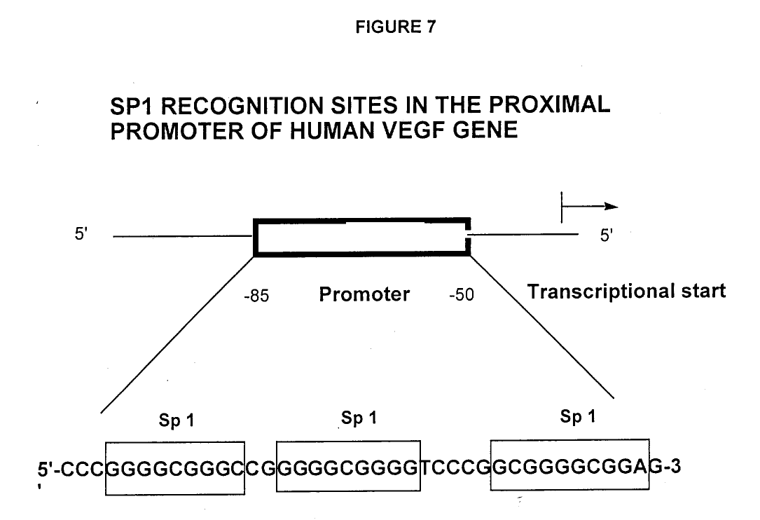

Nucleic acids in a chair conformation often have similar but not identical structures when compared to one another and differences can manifest in loop size and nucleotide sequence, quadruplex geometry, quadruplex stability, the number of alternative chair structures for a given nucleotide sequence, and chemical reactivity, and binding of other molecules. Polymerase arrest assays and chemical footprinting studies applied to the CMYC chair quadruplex structure as described hereafter also were applied to nucleotide sequences upstream of the PDGFA and RET genes. It was determined that the NHE regions 5′ of the RET and PDGFA genes formed chair quadruplex structures that regulated transcription. FIG. 6A depicts a generalized quadruplex chair structure formed in the CMYC, PDGFA, and RET regulatory regions. In this chair structure, CMYC and RET have two stable tetrads and PDGFA has three stable tetrads. The N1 loop for CMYC is TG, for RET is GCG, and for PDGFA is CG. The N2 loop for CMYC is GAG, for RET is GCG, and for PDGFA is GCG. The N3 loop for CMYC is TG, for RET is GCG, and for PDGFA is CG (form A) or AG (form B). The different forms of the PDGFA quadruplex are depicted in FIG. 6C and the RET quadruplex structure is depicted in FIG. 6B. CMYC and PDGFA sequences form stable quadruplex structures under physiological conditions, while the RET sequence required another quadruplex stabilizing molecule for stabilization, such as telomestatin. While CMYC and RET sequences form one chair structure, PDGFA can form two alternative chair structures (form A and form B) that are in equilibrium with one another. All of the stable tetrads in the CMYC, RET, and PDGFA quadruplexes were protected from DMS cleavage and the 3′ G in loop N2 is hypersensitive to DMS cleavage in all three quadruplex structures. While telomestatin stabilized the quadruplex structures of CMYC, RET, and PDGFA, TMPyP4 stabilized only the CMYC and PDGFA quadruplex structures and not the RET quadruplex.

### Substantially Identical Nucleotide Sequences

Nucleotide sequences that are substantially identical to native quadruplex-forming nucleotide sequences are included herein. The term “substantially identical” refers to two or more nucleic acids sharing one or more identical nucleotide sequences. Included are nucleotide sequences that sometimes are 55%, 60%, 65%, 70%, 75%, 80%, or 85% identical to a native quadruplex-forming nucleotide sequence, and often are 90% or 95% identical to the native quadruplex-forming nucleotide sequence (each identity percentage can include a 1%, 2%, 3% or 4% variance). One test for determining whether two nucleic acids are substantially identical is to determine the percentage of identical nucleotide sequences shared between the nucleic acids.

Calculations of sequence identity can be performed as follows. Sequences are aligned for optimal comparison purposes and gaps can be introduced in one or both of a first and a second nucleic acid sequence for optimal alignment. Also, non-homologous sequences can be disregarded for comparison purposes. The length of a reference sequence aligned for comparison purposes sometimes is 30% or more, 40% or more, 50% or more, often 60% or more, and more often 70%, 80%, 90%, 100% of the length of the reference sequence. The nucleotides at corresponding nucleotide positions then are compared among the two sequences. When a position in the first sequence is occupied by the same nucleotide as the corresponding position in the second sequence, the nucleotides are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent conditions is hybridization in 6× sodium chloride/sodium citrate (SSC) at about 45° C., followed by one or more washes in 0.2×SSC, 0.1% SDS at 50° C. Another example of stringent conditions are hybridization in 6× sodium chloride/sodium citrate (SSC) at about 45° C., followed by one or more washes in 0.2×SSC, 0.1% SDS at 55° C. A further example of stringent conditions is hybridization in 6× sodium chloride/sodium citrate (SSC) at about 45° C., followed by one or more washes in 0.2×SSC, 0.1% SDS at 60° C. Often, stringent conditions are hybridization in 6× sodium chloride/sodium citrate (SSC) at about 45° C., followed by one or more washes in 0.2×SSC, 0.1% SDS at 65° C. Also, stringency conditions include hybridization in 0.5M sodium phosphate, 7% SDS at 65° C., followed by one or more washes at 0.2×SSC, 1% SDS at 65° C.

Also, nucleotide sequences of native quadruplex-forming nucleotide sequences may be used as “query sequences” to perform a search against public databases to identify related sequences that may function as aptamer oligonucleotides. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences from FIG. 1. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see, http address www.ncbi.nlm.nih.gov).

### Identification of Quadruplex-Destabilizing Variants and Cancer Prognostics and Diagnostics

Specific genetic alterations are associated with a risk of developing or having certain cancers and/or related disorders. These genetic alterations often are located at polymorphic sites that destabilize quadruplex structures (e.g., the chair structure). As used herein, the term “polymorphic site” refers to a region in a nucleic acid at which two or more alternative nucleotide sequences are observed, often in a significant number of nucleic acid samples from a population of individuals. As described above, these genetic alterations occur at polymorphic sites that destabilize quadruplex structures, and often are nucleotide substitutions from guanine to another nucleotide (e.g., adenine). A polymorphic site often is one nucleotide in length, which is referred to herein as a “single nucleotide polymorphism” or “SNP.” A polymorphic site also may be a nucleotide sequence of two or more nucleotides, an inserted nucleotide or nucleotide sequence, a deleted nucleotide or nucleotide sequence, or a microsatellite, for example.

Where there are two, three, or four alternative nucleotide sequences at a polymorphic site, each nucleotide sequence is referred to as a “mutant sequence,” “substituted sequence,” “polymorphic variant,” “nucleic acid variant,” or “allelic variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess two chromosomes where one is a near copy of the other (e.g., humans). Those individuals who possess the same allelic variants often are referred to as being “homozygous” and those individuals who possess different allelic variants normally are referred to as being “heterozygous.” Homozygous individuals sometimes are predisposed to a different phenotype as compared to heterozygous individuals. As used herein, the term “phenotype” refers to a trait which can be compared between individuals, such as presence or absence of a condition, a visually observable difference in appearance between individuals, a metabolic variation, a physiological variation, a variation in the function of a biological molecule, and the like. An example of a phenotype is occurrence of colorectal cancer.

The term “genotype” refers to a representation of an allelic variant in a subject of a population and the term “genotyped” refers to a method of detecting the presence or absence of a particular allelic variant in a subject of a population. A genotype or polymorphic variant may be expressed in terms of a “haplotype,” which as used herein refers to two or more polymorphic variants occurring on the same chromosome in a group of individuals within a population. For example, two SNPs may exist within a nucleotide sequence where each SNP position includes a cytosine variation and an adenine variation. Certain individuals in a population may carry one allele (heterozygous) or two alleles (homozygous) having a cytosine at each SNP position. As the two cytosines corresponding to each SNP in the gene travel together on one or both alleles in these individuals, the individuals can be characterized as having a cytosine/cytosine haplotype with respect to the two SNPs in the gene.

A polymorphic variant can be identified in any type of nucleic acid sample from any type of biological tissue or fluid. A nucleic acid sample typically is isolated from a biological sample obtained from a subject, and in specific embodiments, subjects diagnosed with cancer. For example, a nucleic acid sample can be isolated from blood, saliva, sputum, and urine, and often is isolated from a cell scraping or biopsy tissue sample (e.g. colorectal tissue) isolated from a subject having cancer. The nucleic acid sample can be isolated from a biological sample using standard techniques, such as described in Example 5. As used herein, the term “subject” primarily refers to humans but also sometimes refers to other mammals such as dogs, cats, and ungulates (e.g. cattle, sheep, and swine). Subjects also sometimes include avians (e.g. chickens and turkeys), reptiles, and fish (e.g. salmon), as methods described herein can be adapted to nucleic acid samples isolated from any of these organisms. The nucleic acid sample may be isolated from the subject and then directly utilized in a method for determining the presence of an allelic variant, or alternatively, the sample may be isolated and then stored (e.g. frozen) for a period of time before being subjected to analysis.

The presence or absence of an allelic variant is detected in one or both chromosomal complements represented in the nucleic acid sample. Determining the presence or absence of a polymorphic variant in both chromosomal complements represented in a nucleic acid sample is useful for determining the zygosity of the polymorphic variant (i.e. whether the subject is homozygous or heterozygous for the polymorphic variant). Any detection method known in the art may be utilized to determine whether a sample includes the presence or absence of a polymorphic variant described herein. While many detection methods include a process in which a DNA region carrying the polymorphic site of interest is amplified, ultrasensitive detection methods which do not require amplification may be utilized in the detection method, thereby eliminating the amplification process. Allelic variant detection methods known in the art include,, for example, nucleotide sequencing methods (see e.g. Example 5); primer extension methods (U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; 5,547,835; 5,605,798; 5,691,141; 5,849,542; 5,869,242; 5,928,906; 6,043,031; 6,194,144; and 6,258,538; WO 01/20039; Chen & Kwok, Nucleic Acids Research 25: 347-353 (1997) and Chen et al., Proc. Natl. Acad. Sci. USA 94/20: 10756-10761 (1997)); ligase sequence determination methods (e.g., U.S. Pat. Nos. 5,679,524 and 5,952,174, and WO 01/27326); mismatch sequence determination methods (e.g., U.S. Pat. Nos. 5,851,770; 5,958,692; 6,110,684; and 6,183,958); microarray sequence determination methods; restriction fragment length polymorphism (RFLP) procedures; PCR-based assays (e.g., TAQMAN™ PCR System (Applied Biosystems)); hybridization methods; conventional dot blot analyses; single strand conformational polymorphism analysis (SSCP, e.g., U.S. Pat. Nos. 5,891,625 and 6,013,499; Orita et al., Proc. Natl. Acad. Sci. U.S.A 86: 27776-2770 (1989)); denaturing gradient gel electrophoresis (DGGE); heteroduplex analysis; mismatch cleavage detection; and techniques described in Sheffield et al., Proc. Natl. Acad. Sci. USA 49: 699-706 (1991), White et al., Genomics 12: 301-306 (1992), Grompe et al., Proc. Natl. Acad. Sci. USA 86: 5855-5892 (1989), and Grompe, Nature Genetics 5: 111-117 (1993). Those of skill in the art can utilize the determined nucleotide sequences flanking a polymorphic site in a database search to determine where the polymorphic site is located in genomic DNA.

A microarray can be utilized for determining whether a polymorphic variant is present or absent in a nucleic acid sample. A microarray may include any oligonucleotide useful for detecting a quadruplex-destabilizing allelic variant, and methods for making and using oligonucleotide microarrays suitable for use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and oligonucleotides may be linked to this solid support by covalent bonds or by non-covalent interactions. Oligonucleotides also may be linked to the solid support directly or by a spacer molecule.

In another embodiment, an integrated system is utilized for determining whether a polymorphic variant is present or absent in a nucleic acid sample. An example of an integrated system is a microfluidic system. These systems comprise a pattern of micro channels designed onto a glass, silicon, quartz, or plastic wafer included on a microchip. The movements of the samples are controlled by electric, electroosmotic or hydrostatic forces applied across different areas of the microchip. The microfluidic system may integrate nucleic acid amplification, sequencing, capillary electrophoresis and a detection method such as laser-induced fluorescence detection.

In yet another embodiment, a kit is utilized to identify a genetic alteration in a sample. A kit often comprises one or more oligonucleotides useful for identifying a quadruplex-destabilizing polymorphic variant. Such oligonucleotides may amplify a fragment of genomic DNA having a polymorphic site associated with quadruplex destabilization. The kit sometimes comprises a polymerizing agent, for example, a thermostable nucleic acid polymerase such as one disclosed in U.S. Pat. Nos. 4,889,818 or 6,077,664. Also, the kit often comprises chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain. The kit can include one or more chain terminating nucleotides such as ddATP, ddTTP, ddGTP, ddCTP, and the like. Kits optionally include buffers, vials, microtitre plates, and instructions for use.

In an embodiment, tissue samples are isolated from subjects diagnosed with a cancer and subjects diagnosed as not having the cancer, a nucleic acid sample is prepared from each tissue sample, and one or more quadruplex-forming nucleotide sequences are analyzed to identify quadruplex-destabilizing nucleotide substitutions associated with the cancer. The cancer can be any cancer, including but not limited to cancer of the colorectum, breast, lung, liver, pancreas, lymph node, colon, prostate, brain, head and neck, skin, liver, kidney, and heart. The isolated tissue is any located at or near the site affected by cancer, and sometimes is from a tumor or polyp, for example. The tissue sample sometimes is frozen, placed in agar, cut into thin slices, and dissected (e.g., with a laser). The quadruplex-forming nucleic acid can have the sequence conforming to the sequence motif described above, or any specific quadruplex nucleotide sequences described herein (e.g., Table 1), and the quadruplex-destabilizing nucleotide substitution can be any described herein. Any of the methods for identifying a nucleotide substitutions described above can be utilized, and a standard nucleotide sequencing procedure preceded by a polymerase chain reaction procedure for amplifying the quadruplex-forming nucleotide sequence in the sample often is utilized, as described in Example 5 in connection with colorectal cancer. A nucleotide substitution is identified as associated with a cancer when it is present in a higher fraction of nucleic acid samples derived from subjects having cancer, and optionally, if the substitution is present in a significant fraction of the nucleic acid samples from subjects having cancer, for example, in nucleic acid samples from 5% or more, 10% or more, 15% or more, 20% or more, 25% or more, 30% or more, 40% or more, or 50% or more of the subjects having cancer.

Cancer prognostic and diagnostic methods generally are directed to detecting the presence or absence of one or more genetic alterations in a nucleic acid sample from a subject, where the presence of a particular genetic alteration determines that the subject is at risk of developing or having a cancer. In specific embodiments, any of the foregoing detection methods may be utilized to prognose or diagnose a cancer associated with quadruplex destabilization by detecting the presence of a quadruplex-destabilizing allelic variant in a nucleic acid sample from a subject. Examples of cancers and related disorders associated with quadruplex destabilization are those associated with deregulation of genes listed in Table 1, such as CMYC, RET, and PDGFA. For example, quadruplex-destabilized deregulation of CMYC is associated with colorectal cancer; deregulation of HER2/neu is associated with breast, pancreatic, and intestinal cancers; deregulation of PDGFA is associated with mesenchymal cell cancers; and deregulation of VEGF is associated with reduced angiogenesis.

In specific embodiments, the risk of a subject developing or having colorectal cancer can be determined by detecting the presence of a specific guanine substitution in the CMYC regulatory sequence TGGGGAGGGTGGGGAGGGTGGGG, where specific guanines are underlined and often are replaced by adenine. For a sequence complementary to the foregoing sequence, detecting a thymidine that replaces a cytosine corresponding to an underlined guanine also is probative of colorectal cancer risk. It should be noted that one or both of the underlined guanines may be replaced with an adenine or another nucleotide, and detection of such replacements is indicative of an increased risk of colorectal cancer. A subject may be heterozygous or homozygous with respect to the quadruplex-destabilizing allele. A subject homozygous for the quadruplex-destabilizing allele normally is at an increased risk of colorectal cancer as compared to a subject homozygous for such an allele.

Predisposition to cancer or a related disorder can be expressed as a probability, such as an odds ratio, percentage, or risk factor. The predisposition is based upon the presence or absence of one or more quadruplex-destabilizing alleles, and also may be based in part upon phenotypic traits of the individual being tested. Methods for calculating risk factors based upon patient data are well known (see e.g. Agresti, Categorical Data Analysis, 2nd Ed. 2002. Wiley).

Results from prognostic and diagnostic tests may be combined with other test results to diagnose, prevent, and treat cancer, as described in greater detail hereafter. Cancer prognostic and diagnostic methods tests sometimes are applied to nucleic acid samples derived from different subjects having varying stages of a particular cancer and sometimes are applied to nucleic acid samples derived from tissue samples representative of varying stages of a particular sample. In colorectal cancer, for example, the presence of a quadruplex-destabilizing allelic variant is detected in nucleic acid samples corresponding to different stages of colorectal cancer and the presence of the allelic variant then is associated with one or more stages of the cancer for a diagnostic test.

### Applications of Prognostic and Diagnostic Test Results

Pharmacogenomics is a discipline that involves tailoring a treatment for a subject according to the subject's genotype, as a particular treatment regimen may exert a differential effect depending upon the subject's genotype. Based upon the outcome of a prognostic or diagnostic test described herein, a clinician or physician may target a preventative or therapeutic treatment to a subject who would be benefited and avoid directing such a treatment to a subject who would not be benefited (e.g., the treatment has no therapeutic effect and/or the subject experiences adverse side effects).

The prognostic and diagnostic methods described herein are applicable to methods for preventing and treating cancer. For example, a nucleic acid sample from an individual may be subjected to a prognostic/diagnostic test described herein. Where one or more quadruplex-destabilizing alleles associated with increased risk of cancer are identified in that subject, other diagnostic methods then may be ordered to characterize the progression of the cancer, and/or one or more cancer preventative regimens or treatment regimens then may be prescribed to that subject. The cancer preventative regimen or treatment regimen may be a general anticancer therapeutic (e.g. chemotherapeutic) or be allele-specific (e.g. antisense therapeutic).

For example, a subject identified by the prognostic or diagnostic procedures described above as having a specific guanine in the sequence TGGGGAGGGTGGGGAGGGTGGGG (each specific guanine is underlined) replaced by an adenine or another nucleotide is identified as being at risk of developing or having colorectal cancer, and a colorectal scope procedure then may be ordered. In the event the scoping procedure identifies only polyps, the polyps may be removed surgically and early removal of any polyps decreases the probability that a more advanced stage of colorectal cancer manifests. Also, a biopsy or tissue scraping procedure may be prescribed and the tissue sample can be analyzed for the presence of cancerous cells. Thus, such a method allows for early detection and prevention of colorectal cancer.

In the event that a quadruplex-destabilizing allele is detected, a colorectal scope procedure is ordered and completed, and colorectal tumors are detected by the scope procedure, a therapeutic treatment regimen for removing, shrinking or minimizing colorectal tumor growth can be prescribed to the subject. Such therapeutic treatment regimens include surgical removal of a tumor or tumors, chemotherapy, and/or radiation treatment, and these therapies can be carried out in any order or combination. For example, surgical removal often is followed by chemotherapy (e.g. fluorouracil, irinotecan, oxaliplatin), and sometimes chemotherapy is used in combination with radiation therapy to decrease colorectal tumor size before surgical removal of the tumor. These strategies are employed for earlier treatment of colorectal cancer, thereby enhancing the possibility of recovery.

In addition to the general therapeutic treatment regimens described above, allele-specific treatment regimens also may proscribed to subjects determined to require the therapeutic based upon prognostic or diagnostic test results. In certain embodiments, a prognostic or diagnostic test described herein is used to detect a quadruplex-destabilizing cancer associated allele in the DNA of a subject, and for subjects having a cancer associated allele, a molecule that interacts and often specifically interacts with the quadruplex-destabilized nucleic acid is administered to the subject. In an embodiment, a peptide nucleic acid (PNA) molecule that specifically hybridizes to the cancer-associated allele is administered to the subject to treat the cancer, as described in greater detail hereafter.

### Quadruplex-Interacting Molecules

Native quadruplex nucleic acids and variants thereof (e.g. a nucleic acid having a quadruplex-destabilizing mutation) are utilized to screen for molecules that specifically interact with quadruplex structures. In these screening assays, one or more candidate molecules (also referred to as “test molecules” or “test compounds”) may be added to a system, where test molecules and quadruplex nucleic acids can be added to the system in any order. For example, a test molecule may be added to a system after a nucleic acid is added; a test molecule may be added to a system before a nucleic acid is added; or a test molecule may be added simultaneously to a system with a nucleic acid. A quadruplex nucleic acid often is added to a system and then a test molecule is added.

Quadruplex interacting molecules typically interact with quadruplexes by reversible binding, and can stabilize already formed quadruplex structures or act as a template for generating quadruplex structures. Quadruplex interacting molecules often exhibit a hyperbolic relationship when biological activity is plotted as a function of quadruplex interacting molecule concentration. The quadruplex interacting molecule sometimes increases or decreases the biological activity being monitored. In addition to reversible binding, test molecules may interact with nucleic acids with irreversible binding, by cleaving one or more strands of a nucleic acid, or by adding chemical moieties to the nucleic acid (e.g., alkylation), for example, depending upon the structure and function of the test molecule.

Test molecules sometimes are organic or inorganic compounds having a molecular weight of 10,000 grams per mole or less, and sometimes having a molecular weight of 5,000 grams per mole or less, 1,000 grams per mole or less, or 500 grams per mole or less. Also included are salts, esters, and other pharmaceutically acceptable forms of the compounds. Compounds that interact with nucleic acids are known in the art (see, e.g. Hurley, Nature Rev. Cancer 2, 188-200 (2002); Anantha et al., Biochemistry Vol. 37, No. 9: 2709-2714 (1998); and Ren et al., Biochemistry 38: 16067-16075 (1999)).

Compounds can be obtained using any of the combinatorial library methods known in the art, including spatially addressable parallel solid phase or solution phase libraries; synthetic library methods requiring deconvolution; “one-bead one-compound” library methods; and synthetic library methods using affinity chromatography selection. Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and Gallop et al., J. Med. Chem. 37: 1233 (1994).

In addition to an organic and inorganic compound, a test molecule is sometimes a nucleic acid, an antisense nucleic acid (described in more detail hereafter), a catalytic nucleic acid (e.g., a ribozyme), a nucleotide, a nucleotide analog, a polypeptide, an antibody, or a peptide mimetic. Methods for making and using these test molecules are known in the art. For example, methods for making ribozymes and assessing ribozyme activity are described (see, e.g., U.S. Pat. Nos. 5,093,246; 4,987,071; and 5,116,742; Haselhoff & Gerlach, Nature 334: 585-591 (1988) and Bartel & Szostak, Science 261: 1411-1418 (1993)). Also, peptide mimetic libraries are described (see, e.g., Zuckermann et al., J. Med. Chem. 37: 2678-85 (1994)).

### Systems and Solid Supports

In assays that detect the presence or absence of an interaction between a quadruplex nucleic acid and a quadruplex-interacting molecule, test molecules are contacted with a nucleic acid in a system. As used herein, the term “contacting” refers to placing a signal molecule and/or a test molecule in close proximity to a quadruplex nucleic acid or test nucleic acid and allowing the molecules to collide with one another by diffusion. Contacting these assay components with one another can be accomplished by adding assay components to one body of fluid or in one reaction vessel, for example. The components in the system may be mixed in variety of manners, such as by oscillating a vessel, subjecting a vessel to a vortex generating apparatus, repeated mixing with a pipette or pipettes, or by passing fluid containing one assay component over a surface having another assay component immobilized thereon, for example.

As used herein, the term “zsystem” refers to an environment that receives the assay components, which includes, for example, microtitre plates (e.g., 96-well or 384-well plates), silicon chips having molecules immobilized thereon and optionally oriented in an array (e.g., described above and in U.S. Pat. No. 6,261,776 and Fodor, Nature 364: 555-556 (1993)), and microfluidic devices (e.g., described above and in U.S. Pat. Nos. 6,440,722; 6,429,025; 6,379,974; and 6,316,781). The system can include attendant equipment for carrying out the assays, such as signal detectors, robotic platforms, and pipette dispensers.

One or more assay components may be immobilized to a solid support. The attachment between an assay component and the solid support may be covalent or non-covalent (see, e.g., U.S. Pat. No. 6,022,688 for non-covalent attachments). The solid support may be one or more surfaces of the system, such as one or more surfaces in each well of a microtiter plate, a surface of a silicon wafer, a surface of a bead (see e.g. Lam, Nature 354: 82-84 (1991)) that is optionally linked to another solid support, or a channel in a microfluidic device, for example. Types of solid supports, linker molecules for covalent and non-covalent attachments to solid supports, and methods for immobilizing nucleic acids and other molecules to solid supports are known (see e.g. U.S. Pat. Nos. 6,261,776; 5,900,481; 6,133,436; and 6,022,688; and WIPO publication WO 01/18234).

In an embodiment, polypeptide test molecules may be linked to a phage via a phage coat protein. The latter embodiment is often accomplished by using a phage display system, where quadruplex nucleic acids linked to a solid support are contacted with phages that display different polypeptide test molecules. Phages displaying polypeptide test molecules that interact with the immobilized nucleic acids adhere to the solid support, and phage nucleic acids corresponding to the adhered phages are then isolated and sequenced to determine the sequence of the polypeptide test molecules that interacted with the immobilized nucleic acids. Methods for displaying a wide variety of peptides or proteins as fusions with bacteriophage coat proteins are well known (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991)). Methods are also available for linking the test polypeptide to the N-terminus or the C-terminus of the phage coat protein. The original phage display system was disclosed, for example, in U.S. Pat. Nos. 5,096,815 and 5,198,346. This system used the filamentous phage M13, which required that the cloned protein be generated in E. coli and required translocation of the cloned protein across the E. coli inner membrane. Lytic bacteriophage vectors, such as lambda, T4 and T7 are more practical since they are independent of E. coli secretion. T7 is commercially available and described in U.S. Pat. Nos. 5,223,409; 5,403,484; 5,571,698; and 5,766,905.

### Identifying Quadruplex Interacting Molecules

Test molecules often are identified as quadruplex interacting molecules where a biological activity of the quadruplex, often expressed as a “signal,” produced in a system containing the test molecule is different than the signal produced in a system not containing the test molecule. Also, test nucleic acids are identified as quadruplex forming nucleic acids when the signal detected in a system that includes the test nucleic acid is different than the signal detected in a system that does not include the test nucleic acid. While background signals may be assessed each time a new molecule is probed by the assay, detecting the background signal is not required each time a new molecule is assayed.

In addition to determining whether a test molecule or test nucleic acid gives rise to a different signal, the affinity of the interaction between the nucleic acid and test molecule or signal molecule may be quantified. IC₅₀, K_{d}, or Kᵢ threshold values may be compared to the measured IC₅₀ or K_{d} values for each interaction, and thereby identify a test molecule as a quadruplex interacting molecule or a test nucleic acid as a quadruplex forming nucleic acid. For example, IC₅₀ or K_{d} threshold values of 10 μM or less, 1 μM or less, and 100 nM or less are often utilized, and sometimes threshold values of 10 nM or less, 1 nM or less, 100 pM or less, and 10 pM or less are utilized to identify quadruplex interacting molecules and quadruplex forming nucleic acids.

Many assays are available for identifying quadruplex interacting molecules and quadruplex forming nucleic acids. In some of these assays, the biological activity is the quadruplex nucleic acid binding to a molecule and binding is measured as a signal. In other assays, the biological activity is a polymerase arresting function of a quadruplex and the degree of arrest is measured as a decrease in a signal. In certain assays, the biological activity is transcription and transcription levels can be quantified as a signal. In another assay, the biological activity is cell death and the number of cells undergoing cell death is quantified. Another assay monitors proliferation rates of cancer cells. Examples of assays are fluorescence binding assays, gel mobility shift assays (see, e.g., Jin & Pike, Mol. Endocrinol. 10: 196-205 (1996)), polymerase arrest assays, transcription reporter assays, cancer cell proliferation assays, and apoptosis assays (see, e.g., Amersham Biosciences (Piscataway, N.J.)), and embodiments of such assays are described hereafter and in Example 8. Also, topoisomerase assays can be utilized to determine whether the quadruplex interacting molecules have a topoisomerase pathway activity (see e.g. TopoGEN, Inc. (Columbus, Ohio)).

An example of a fluorescence binding assay is a system that includes a quadruplex nucleic acid, a signal molecule, and a test molecule. The signal molecule generates a fluorescent signal when bound to the quadruplex nucleic acid (e.g. N-methylmesoporphyrin IX (NMM)), and the signal is altered when a test molecule competes with the signal molecule for binding to the quadruplex nucleic acid. An alteration in the signal when test molecule is present as compared to when test molecule is not present identifies the test molecule as a quadruplex interacting molecule.

An example of an arrest assay is a system that includes a template nucleic acid, which may comprise a quadruplex forming sequence, and a primer nucleic acid which hybridizes to the template nucleic acid 5′ of the quadruplex-forming sequence. The primer is extended by a polymerase (e.g., Taq polymerase), which advances from the primer along the template nucleic acid. In this assay, a quadruplex structure can block or arrest the advance of the enzyme, leading to shorter transcription fragments. Also, the arrest assay may be conducted at a variety of temperatures, including 45° C. and 60° C., and at a variety of ion concentrations.

In a transcription reporter assay, test quadruplex DNA may be coupled to a reporter system, such that a formation or stabilization of a quadruplex structure can modulate a reporter signal. An example of such a system is a reporter expression system in which a polypeptide, such as luciferase or green fluorescent protein (GFP), is expressed by a gene operably linked to the potential quadruplex forming nucleic acid and expression of the polypeptide can be detected. As used herein, the term “operably linked” refers to a nucleotide sequence which is regulated by a sequence comprising the potential quadruplex forming nucleic acid. A sequence may be operably linked when it is on the same nucleic acid as the quadruplex DNA, or on a different nucleic acid. An exemplary luciferase reporter system is described herein.

In a cancer cell proliferation assay, cell proliferation rates are assessed as a function of different concentrations of test quadruplex interacting molecules added to the cell culture medium. Any cancer cell type can be utilized in the assay. In one embodiment, colon cancer cells are cultured in vitro and test quadruplex-interacting molecules are added to the culture medium at varying concentrations. A useful colon cancer cell line is colo320, which is a colon adenocarcinoma cell line deposited with the National Institutes of Health as accession number JCRB0225. Parameters for using such cells are available at the http address cellbank.nihs.go.jp/cell/data/jcrb0225.htm.

### Nucleic Acid Therapeutics Targeted to Quadruplex-Destabilizing Alleles

Provided herein are nucleic acid therapeutics that specifically interact with quadruplex-destabilizing alleles. In one embodiment, antisense nucleic acids are designed to hybridize to quadruplex-destabilized alleles. The nucleotide sequence of the antisense nucleic acid is designed around one or more polymorphic sites associated with quadruplex-destabilization. For example, upon identification of a quadruplex-destabilizing mutation in any of the native quadruplex nucleic acids described above, a nucleic acid complementary to the quadruplex-destabilized nucleic acid is generated that includes a sequence complementary to the polymorphic site (e.g. if the polymorphic site is a guanine to adenine base mutation, the complementary nucleic acid includes a thymine designed to hybridize to the adenine mutation and other nucleotides flanking the 5′ and/or 3′ end of the thymine). In an embodiment, a colorectal cancer therapeutic nucleic acid has a nucleotide sequence that hybridizes to one of the following CMYC quadruplex-destabilizing cancer-associated alleles: TGGGGAGGGTGXGGAGGGTGGGG, TGGGGAGGGTGGGGAGXGTGGGG, or TGGGGAGGGTGXGGAGXGTGGGG, where G is guanine, A is adenine, C is cytosine, T is thymine, and X is any nucleotide except for guanine. Thus, the colorectal cancer therapeutic nucleic acid may comprise all or part of the nucleotide sequences CCCCACCCTCCYCACCCTCCCCA, CCCCACYCTCCCCACCCTCCCCA, or CCCCACYCTCCYCACCCTCCCCA, where G is guanine, A is adenine, C is cytosine, T is thymine, and Y is a nucleotide complementary to X (e.g. the nucleic acid may be 20 nucleotides in length and include a portion of the sequence that includes the nucleotide complementary to the polymorphic site). The therapeutic nucleic acid may be any length that allows hybridization to the target nucleotide sequence in vivo. The nucleic acid therapeutics sometimes are about 7, about 8, about 9, or about 10 nucleotides in length, often are about 12 or fewer, about 15 or fewer, about 17 or fewer, or about 20 or fewer nucleotides in length, and sometimes are about 25 or fewer, about 30 or fewer, about 40 or fewer, or about 50 or fewer nucleotides in length.

The quadruplex-targeted nucleic acid often is synthesized having a backbone with fewer negative charges as compared to a DNA backbone. Examples of such nucleic acids are peptide nucleic acids (PNA) and PNA molecules having amino acid side chain moieties (e.g. lysine, arginine, and histidine side chain moieties (see e.g. U.S. patent application publication no. 20020188101 (Neilsen et al.)). In an embodiment, the nucleic acid is a PNA optionally linked at the C-terminus to a lysine moiety. In another embodiment, the PNA is conjugated to another peptide that facilitates transduction of the conjugate into cells. Examples of such transduction peptides are HIV tat peptides (see e.g. SEQ ID NOs: 2-7 of U.S. Pat. No. 5,652,122) and Antennapedia homeodomain peptides (e.g. GGRQIWFQNRMKWKK, GGLWFQNRMKWKKEN, GGGRQIKIWFQNRRMKWKK, or GGGKIWFQNRRMKWKKEN reported in Simmons et al., Bioorg. Med. Chem. Lttrs. 7: 3001-3006 (1997)). The transduction peptide is linked to the N-terminal or C-terminal end of the DNA using standard techniques. When the peptide is attached to the C-terminus of the PNA, the PNA often will not include a C-terminal lysine moiety.

The quadruplex-targeted nucleic acid often is tested in vitro to determine the degree to which it hybridizes to a nucleic acid corresponding to a native quadruplex nucleotide sequence or a quadruplex-destabilizing allele. A fluorescence binding assay or circular dichroism assay (see e.g. Example 8) can be utilized to determine whether the quadruplex-targeted nucleic acid hybridizes to the target nucleic acid. Also, the colo320 cell line described above can be utilized to determine whether the PNA exerts a biological effect, for example, on cell proliferation. In specific embodiments, the target nucleic acid is TGGGGAGGGTGGGGAGGGTGGGG, TGGGGAGGGTGXGGAGGGTGGGG, TGGGGAGGGTGGGGAGXGTGGGG, or TGGGGAGGGTGXGGAGXGTGGGG, where G is guanine, A is adenine, C is cytosine, T is thymine, and X is any nucleotide except for guanine. Upon a determination that the quadruplex targeting nucleic acid is functional in vitro, the nucleic acid often is screened in vivo in animal models or in human subjects and the effect on cancer is monitored.

The quadruplex-targeted nucleic acid can be administered in vitro or in vivo as a composition of a pharmaceutically acceptable salt, ester, or salt of such ester. The quadruplex-targeted nucleic acid can be formulated as naked polynucleotide (e.g. polynucleotide formulated in phosphate buffered saline) or it can be formulated with other components.

Compositions comprising a quadruplex-targeted nucleic acid can be prepared as a solution, emulsion, or polymatrix-containing formulation (e.g., liposome and microsphere). Examples of such compositions are set forth in U.S. Pat. Nos. 6,455,308 (Freier), 6,455,307 (McKay et al.), 6,451,602 (Popoff et al.), and 6,451,538 (Cowsert), and examples of liposomes also are described in U.S. Pat. No. 5,703,055 (Felgner et al.) and Gregoriadis, Liposome Technology vols. I to III (2nd ed. 1993). The compositions can be prepared for any mode of administration, including topical, oral, pulmonary, parenteral, intrathecal, and intranutrical administration. Examples of compositions for particular modes of administration are set forth in U.S. Pat. Nos. 6,455,308 (Freler), 6,455,307 (McKay et al.), 6,451,602 (Popoffet al.), and 6,451,538 (Cowsert). Quadruplex-targeted nucleic acid compositions may include one or more pharmaceutically acceptable carriers, excipients, penetration enhancers, and/or adjuncts. Choosing the combination of pharmaceutically acceptable salts, carriers, excipients, penetration enhancers, and/or adjuncts in the composition depends in part upon the mode of administration. Guidelines for choosing the combination of components for a quadruplex-targeted nucleic acid composition are known, and examples are set forth in U.S. Pat. Nos. 6,455,308 (Freier), 6,455,307 (McKay et al.), 6,451,602 (Popoff et al.), and 6,451,538 (Cowsert).

A quadruplex-targeted nucleic acid in the composition may be modified by chemical linkages, moieties, or conjugates that enhance activity, cellular distribution, or cellular uptake of the nucleic acid. Examples of such modifications are set forth in U.S. Pat. Nos. 6,455,308 (Freier), 6,455,307 (McKay et al.), 6,451,602 (Popoffet al.), and 6,451,538 (Cowsert).

A quadruplex-targeted nucleic acid compositions may be presented conveniently in unit dosage form, which is prepared according to conventional techniques known in the pharmaceutical industry. In general terms, such techniques include bringing a quadruplex-targeted nucleic acid into association with pharmaceutical carrier(s) and/or excipient(s) in liquid form or finely divided solid form, or both, and then shaping the product if required. The quadruplex-targeted nucleic acid compositions may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

A quadruplex-targeted nucleic acid can be translocated into cells via conventional transformation or transfection techniques. As used herein, the terms “transformation” and “transfection” refer to a variety of standard techniques for introducing an aptamer into a host cell, which include calcium phosphate or calcium chloride co-precipitation, transduction/infection, DEAE-dextran-mediated transfection, lipofection, electroporation, and iontophoresis. Also, liposome compositions described herein can be utilized to facilitate quadruplex-targeted nucleic acid administration. A quadruplex-targeted nucleic acid composition may be administered to an organism in a number of manners, including topical administration (including ophthalmic and mucous membrane delivery (e.g., vaginal and rectal)), pulmonary administration (e.g., inhalation or insufflation of powders or aerosols, including by nebulizer; intratracheal, intranasal, epidermal and transdermal), oral administration, and parenteral administration (e.g., intravenous, intraarterial, subcutaneous, intraperitoneal injection or infusion, intramuscular injection or infusion; and intracranial (e.g., intrathecal or intraventricular)). In an embodiment, the composition is administered by colorectal delivery.

### Utilization of Quadruplex-Interacting Molecules

Because quadruplex forming nucleic acids are regulators of biological processes such as oncogene transcription, modulators of quadruplex biological activity can be utilized as cancer therapeutics. For example, molecules that interact with quadruplex structures can exert a therapeutic effect for certain cell proliferative disorders and related conditions because abnormally increased oncogene expression can cause cell proliferative disorders and quadruplex structures typically down-regulate oncogene expression. Quadruplex-interacting molecules can exert a biological effect according to different mechanisms, which include for example stabilizing a native quadruplex structure, inhibiting conversion of a native quadruplex to duplex DNA by blocking strand cleavage, and stabilizing a native quadruplex structure having a quadruplex-destabilizing nucleotide substitution. Also, administering a quadruplex forming nucleic acid having a similar or identical nucleotide sequence to a native oncogene regulating quadruplex sequence may act as a decoy by competing for cellular molecules that normally up-regulate an oncogene. Thus, quadruplex forming nucleic acids and quadruplex interacting molecules identified by the methods described herein may be administered to cells, tissues, or organisms for the purpose of down-regulating oncogene transcription and thereby treating cell proliferative disorders. The term “treatment” and “therapeutic effect” as used herein refer to reducing or stopping a cell proliferation rate (e.g. slowing or halting tumor growth) or reducing the number of proliferating cancer cells (e.g. removing part or all of a tumor).

Determining whether the biological activity of native quadruplex DNA is modulated in a cell, tissue, or organism can be accomplished by monitoring quadruplex biological activity. Quadruplex biological activity may be monitored in cells, tissues, or organisms, for example, by detecting a decrease or increase of gene transcription in response to contacting the quadruplex DNA with a molecule. Transcription can be detected by directly observing RNA transcripts or observing polypeptides translated by transcripts, which are methods well known in the art.

Quadruplex interacting molecules and quadruplex forming nucleic acids can be utilized to target many cell proliferative disorders. Cell proliferative disorders include, for example, colorectal cancers. Other examples of cancers include hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-97 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. Cell proliferative disorders also include cancers of the colorectum, breast, lung, liver, pancreas, lymph node, colon, prostate, brain, head and neck, skin, liver, kidney, and heart. Quadruplex interacting molecules also can be utilized to target cancer related processes and conditions, such as increased angiogenesis, by inhibiting angiogenesis in a subject.

Administering a molecule to an organism can be accomplished in a number of manners, including intradermal, intramuscular, intravenous, intraperitoneal, and subcutaneous administration. An effective amount of molecule for modulating the biological activity of native quadruplex DNA will depend in part on the molecule composition, the mode of administration, and the weight and general health of the organism, and can generally range from about 1.0 μg to about 5000 μg of peptide for a 70 kg patient. The effective amount can be optimized by determining whether the biological activity of the native quadruplex DNA is modulated in the system.

Thus, provided herein are methods for reducing cell proliferation or for treating or alleviating cell proliferative disorders, which comprise contacting a system having a native quadruplex DNA with a quadruplex interacting molecule or quadruplex forming nucleic acid identified by an assay described herein. The system sometimes is a group of cells or one or more tissues, and often is a subject in need of a treatment of a cell proliferative disorder (e.g., a mammal such as a mouse, rat, monkey, or human). In an embodiment, provided is a method for treating colorectal cancer by administering a CMYC quadruplex-interacting molecule described herein to a subject in need thereof, thereby reducing the colorectal cancer cell proliferation. In another embodiment, provided is a method for inhibiting angiogenesis and optionally treating a cancer associated with angiogenesis, which comprises administering a VEGF quadruplex-interacting molecule to a subject in need thereof, thereby reducing angiogenesis and optionally treating a cancer associated with angiogenesis.

The invention is further illustrated by the following examples which should not be construed as limiting. The contents of the documents cited in this application are incorporated herein by reference.

### EXAMPLES

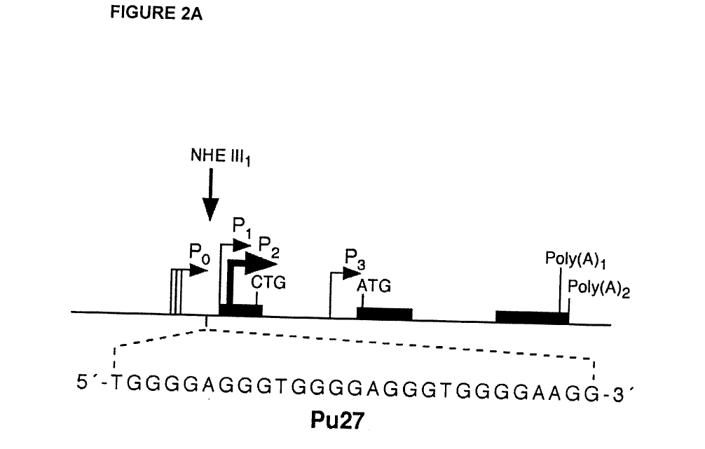

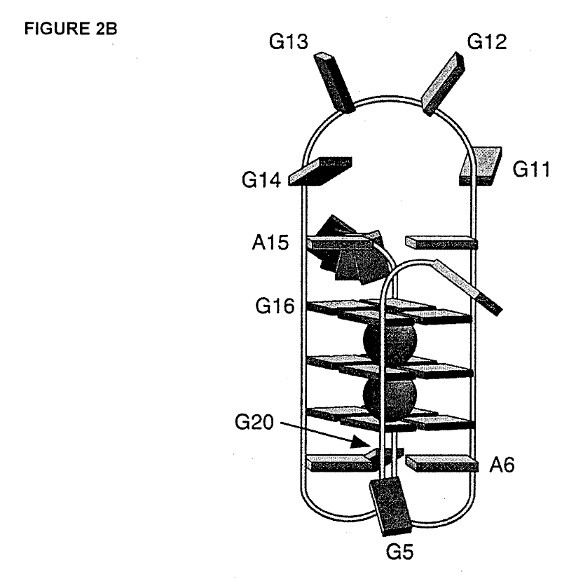

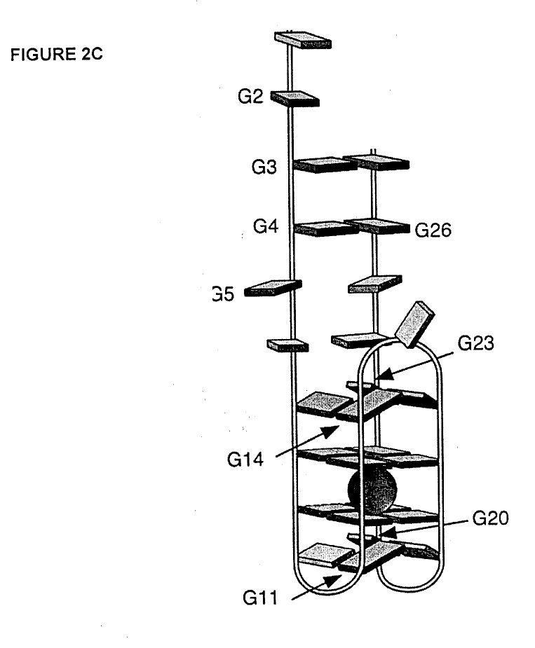

Certain examples were performed in part using a 27 base pair DNA derived from the regulatory region of CMYC, referred to as a nuclease hypersensitivity element III₁ (NHE III₁) (FIG. 2A). The 27 base pair polynucleotide corresponding to the noncoding purine-rich strand of NHE III₁ was synthesized and was referred to as Pu27. It was shown that Pu27 adopted a quadruplex structure and that the biologically significant structure is a chair conformation. It also was shown that alternative nucleotide sequences in this region were associated with colorectal cancer, and colorectal cancer prognostics, diagnostics, and therapeutics were developed accordingly.

### Example 1

### Structural Determination of Pu27 Quadruplex DNA

Pu27 is capable of forming two distinct quadruplex DNA structures. Preparative gel electrophoresis (12.5 mM KCl/NaCl, 16 h, 4° C.) of 3′-end-labeled Pu27 incubated in the presence of 100 mM KCl resulted in the formation of two bands (1 and 2), along with several bands of lower electrophoretic mobility. Bands 1 and 2 were isolated and subjected to DMS-induced strand cleavage. Each band of interest was excised and soaked in 100 mM KCl solution (300 μl) for 6 hr at 4° C. The solutions were filtered (microcentrifuge) and 30,000 cpm (per reaction) of DNA solution was diluted further with 100 mM KCl in 0.1×TE to a total volume of 70 μl (per reaction). Following the addition of 1 μl salmon sperm DNA (0.1 μg/μl), the reaction mixture was subjected to a 1 μl DMS solution (DMS:ethanol; 4:1; v:v) for 5, 10, and sometimes 15 minutes. Each reaction was quenched with 18 μl of stop buffer (β-mercaptoathanol:water:NaOAc (3 M); 1:6:7; v:v:v). Following ethanol precipitation (twice) and piperidine cleavage, the reactions were separated on a preparative gel (16%) and visualized on a phosphorimager. DMS analysis of unstructured Pu27 was performed essentially in the same way, using heat-denatured 3′-end-labeled Pu27 in 0.1×TE buffer. For DMS-cleavage of band 1, each band of interest was excised and soaked in 100 mM KCl solution (300 μl) for 6 hr at 4° C. The solutions were filtered (microcentrifuge) and 30,000 cpm (per reaction) of DNA solution was diluted further with 100 mM KCl in 0.1×TE to a total volume of 70 μl (per reaction). Following the addition of 1 μl salmon sperm DNA (0.1 μg/μl), the reaction mixture was subjected to 1 μl DMS solution (DMS:ethanol; 4:1; v:v) for 5, 10, and sometimes 15 minutes. Each reaction was quenched with 18 μl of stop buffer (β-mercaptoathanol:water:NaOAc (3 M); 1:6:7; v:v:v). Following ethanol precipitation (twice) and piperidine cleavage, the reactions were separated on a preparative gel (16%) and visualized on a phosphorimager. DMS analysis of unstructured Pu27 was performed essentially in the same way, using heat-denatured 3′-end-labeled Pu27 in 0.1×TE buffer.

The pattern of N7 guanine methylation produced by band 1 was consistent with an intramolecular basket quadruplex, which consisted of three stacked G-tetrads, two lateral two-base loops, and a six-base bridging loop (FIG. 2B). For band 2, a distinct and pronounced cleavage pattern suggested the formation of an intramolecular chair quadruplex, comprising two stacked G-tetrads, two lateral two-base loops, and an orthogonal three-base bridging loop (FIG. 2C).

Since G7 and G16 show strong DMS protection relative to the other two guanines immediately above the top G-tetrad (G14 and G23), a staggered, or split, G-tetrad conformation for these four bases is the most probable conformation. Presumably the excessive strain that should be imposed by a one-base bridging loop prevents the adoption of a planar tetrad. DMS footprinting of bands of lower mobility than band 1 were consistent with higher-order quadruplex structures. An identical DMS footprint to that found for band 2 can be produced from Pu27 exposed to 100 mM KCl for 5 min, prior to DMS treatment. Therefore, the chair quadruplex, which results from the simple folding over of a DNA G-hairpin, is kinetically favored, while the basket quadruplex, having a more complex folding pattern associated with higher energy intermediates and a greater number of stacked G-tetrads, is slower to form and is thermodynamically favored. Based upon these studies, the structure proposed for Pu27 is similar to T30695.

### Example 2

### Determination that the Chair Conformation is Biologically Significant

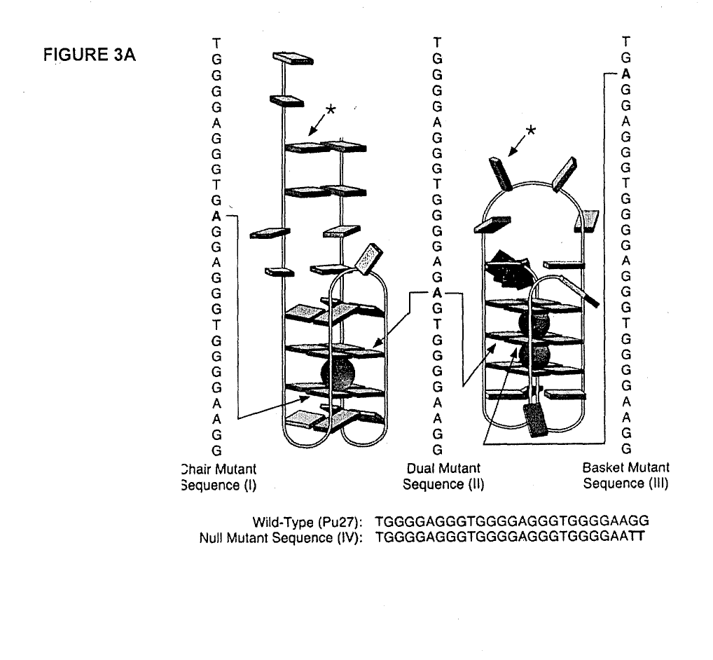

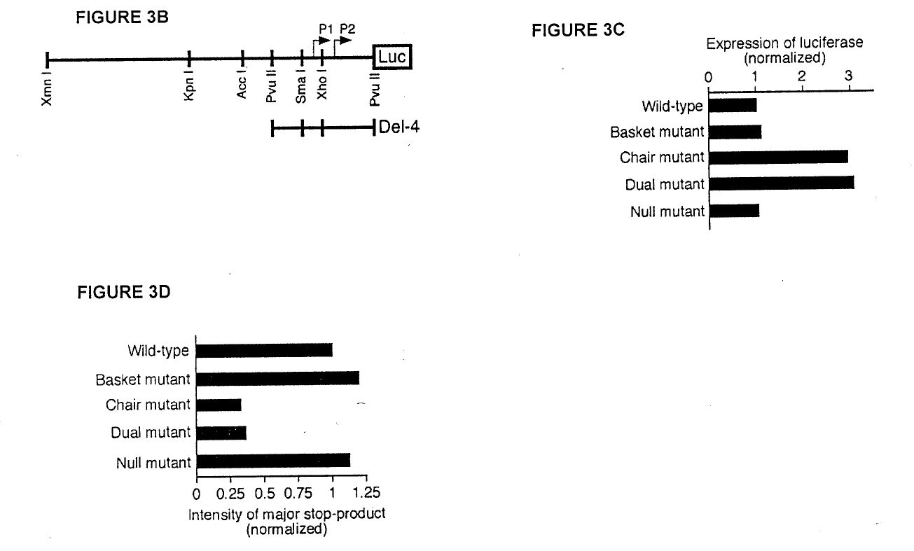

To evaluate the potential biological significance of the two intramolecular G-quadruplex structures, single- or double-base mutations of Pu27 were designed (FIG. 3A) and the various constructs were evaluated for both promoter activity in a luciferase reporter assay (FIGS. 3B and 3C) and for the ability to form stable G-quadruplex structures using a Taq polymerase stop assay (FIG. 3D). Site directed mutants were prepared using standard protocols.

The luciferase promoter assay utilized for this study was described in He T.-C. et al., Science 281: 1509-1512 (1998). Specifically, a vector utilized for the assay is set forth in reference 11 of the He T.-C. et al. document. In this assay, HeLa S₃ cells were transfected using the Effectene lipid-based system (QIAgen) according to the manufacturer's protocol, using 0.1 μg of pRL-TK (Renilla luciferase reporter plasmid) and 0.9 μg of the Del-4 (wild-type) or mutated plasmids (see QIAgen Effectene Transfection Reagent Handbook, March 2001). Firefly and Renilla luciferase activities were assayed using the Dual Luciferase Reporter Assay System (Promega) in a 96-well plate format according to the manufacturer's protocol.

In the luciferase reporter assay, single-base mutations, which destabilized a single tetrad uniquely associated with either the basket or chair G-quadruplex (FIG. 3A), had quite different effects (FIG. 3C). In comparison to the wild-type sequence, a single-base mutation of a tetrad unique to the chair form resulted in a 3-fold increase in transcriptional activity, whereas a corresponding mutation to the basket form had a negligible effect. A single-base mutation, which eliminated a G-tetrad in both chair and basket forms, also caused a 3-fold increase in transcriptional activation, whereas a null double-base mutation, which has no effect on either G-quadruplex structure, had the same activity as the wild-type. These results led to two conclusions: (1) only the chair G-quadruplex structure was biologically relevant and (2) disruption of the chair G-quadruplex structure resulted in a significant increase in transcriptional activation (3-fold), implicating formation of this G-quadruplex structure as a repressor element to transcriptional activation of CMYC.

The same wild-type and mutant sequences of the Pu27 were used in a Taq polymerase stop assay, which was described in Han et al., Nucl. Acids Res. 27: 537-542 (1999). This assay is a modification of that used by Weitzmann et al., J. Biol. Chem. 271, 20958-20964 (1996), and is also described below. A reaction mixture of template DNA (77-mer with a Pu27 or mutant Pu27 insert) (50 nM), Tris.HCl (50 mM), MgCl₂ (10 mM), DTT (0.5 mM), EDTA (0.1 mM), BSA (60 ng), and 5′-end-labeled 18-mer primer (˜18 nM) was heated to 90° C. for 5 min and allowed to cool to ambient temperature over 30 min. Taq Polymerase (1 μl) was added to the reaction mixture, and the reaction was maintained at a constant temperature (45° C. for the mutant Pu27 comparison; 60° C. for the porphyrin comparison) for 30 min. (For the porphyrin comparison, following cooling to ambient temperature, the requisite amount of porphyrin solution was added to the reaction mixture and left at ambient temperature for 30 min prior to the addition of polymerase.) Following the addition of 10 μl stop buffer (formamide [20 ml], 1 M NaOH [200 μl], 0.5 M EDTA [400 μl], 10 mg bromophenol blue), the DNA fragments were separated with a sequencing gel (12%) and visualized on a phosphorimager. Adenine sequencing (indicated by “A” at the top of the gel) was performed using double-stranded DNA Cycle Sequencing System from Life Technologies. The general sequence for 77-mer template strands was TCCAACTATGTATAC-INSERT-TTAGCGACACGCAATTGCTATAGTGAGTCGTATTA, and the 1 8-mer primer had the sequence TAATACGACTCACTATAG.

In the polymerase arrest assay, primer extension using Taq polymerase led to premature stops at G-quadruplex structures. These results showed that significant arrest occurred with the wild-type insert at 45° C. The primary arrest site occurred at G37, which corresponds to G7 in Pu27. The single-base mutant in the basket G-quadruplex and the null double-base mutant were equivalent to the wild-type, while the single-base mutant in the chair and the mutant that affects the stability of both G-quadruplex structures led to loss of the G-quadruplex-mediated polymerase stop (FIG. 3D). This result was complementary to that found in the promoter assay, in which only those mutations that resulted in destabilization of the chair quadruplex resulted in enhancement of CMYC transcriptional activation.

### Example 3

### Interaction of Compounds With Quadruplex DNA Structures in Vitro

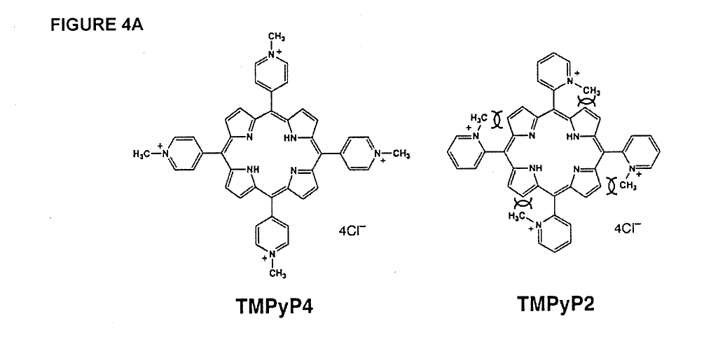

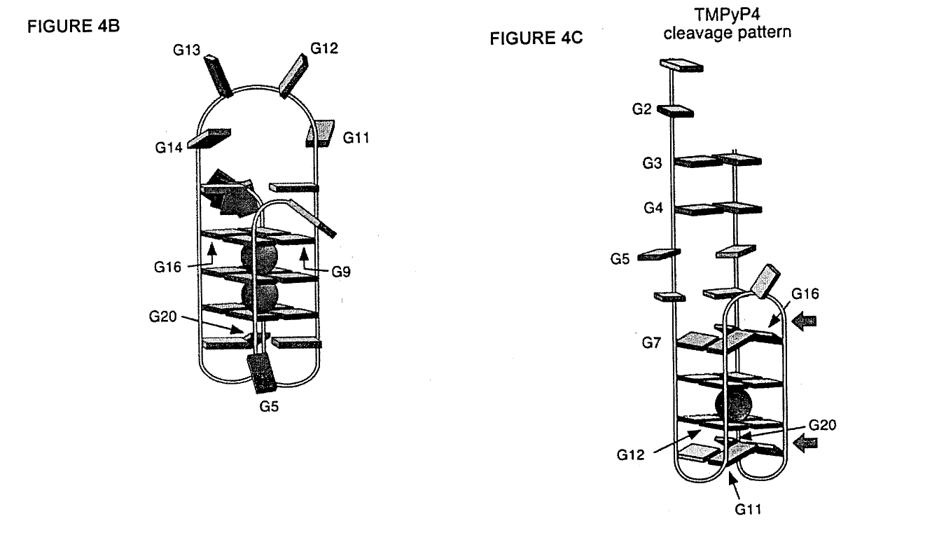

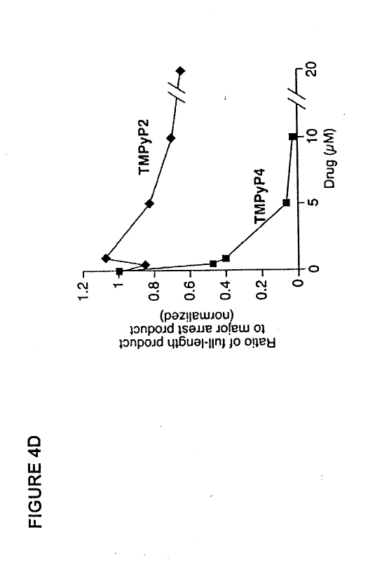

The two quadruplex conformations of Pu27 were contacted with compounds known to bind quadruplex structures. Specifically, the two conformations were contacted with TMPyP4 and TMPyP2 (FIG. 4A) to further confirm the biologically relevant quadruplex conformation (FIGS. 4B or 4C). It has been shown that both TMPyP4 and TMPyP2 catalyze the oxidation of DNA upon exposure to light, which results in DNA strand breakage in proximity to the binding sites (Han et al., J. Am. Chem. Soc. 123: 8902-8913 (2001)). Consequently, the specific cleavage patterns of G-quadruplex DNA by these compounds was utilized to infer their binding modes and sites.

An identical cleavage pattern was produced by both compounds for the basket quadruplex (FIG. 4B). DNA damage was centered on the constituent guanines of the six-base bridging loop, inferring that both porphyrins are able to bind to this region, but in a nonspecific way that is neither intercalating nor end-stacking (FIG. 4A). Conversely, only TMPyP4 gave rise to a specific cleavage pattern when the chair G-quadruplex structure was subjected to photocleavage, which is most likely associated with partial end-stacking of TMPyP4 to the external G-tetrads, that is, at the positions shown by the arrows in FIG. 4C.

To further determine whether this selectivity of TMPyP4 versus TMPyP2 for the chair conformation correlated with an effect on CMYC transcription, a Taq polymerase stop assay was performed at an elevated temperature (60° C.) to partially destabilize the arresting quadruplex structure in the presence of increasing concentrations of TMPyP2 and TMPyP4. Whereas TMPyP2 only modestly stabilized the quadruplex arrest site, even at 20 μM, the addition of TMPyP4 led to almost 65% arrest at 0.5 μM and virtually total arrest at 5 μM. Consequently, these results demonstrated that TMPyP4, but not TMPyP2, exhibited a marked binding and stabilizing effect on the chair quadruplex. This is in agreement with the differential effects of the two compounds on CMYC expression in vitro and sensitivity in vivo to tumors that overexpress CMYC (MX-1 and PC-3).

### Example 4

### Interaction of Compounds With Quadruplex DNA Structures in Cells

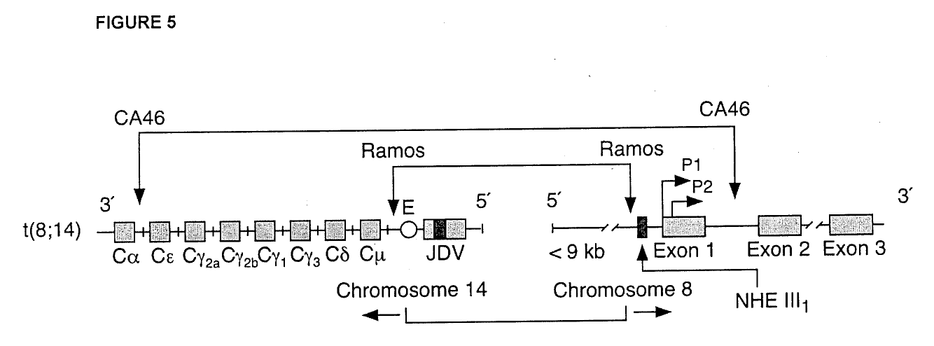

The importance of the NHE III₁ sequence in mediating the CMYC transcriptional inhibition by TMPyP4 was assessed. Two Burkitt's lymphoma cell lines with different translocation break points within the CMYC and immunoglobulin loci were analyzed, which are referred to as the Ramos cell line and the CA46 cell line. While the Ramos cell line retained the NHE III₁ during the translocation, the CA46 cell line lost this element, together with the P1 and P2 promoters (FIG. 5A). When the NHE III₁ element was deleted, as in the CA46 cell line, TMPyP4 had no effect on CMYC transcriptional activation. In the Ramos cell line, however, TMPyP4, but not TMPyP2, lowered CMYC expression. This result is consistent with TMPyP4 mediating its transcriptional inhibitory effect on CMYC by interaction with the NHE III₁ found upstream of the P1 promoter.

The effects of two test molecules also were assessed for the RET quadruplex. MiaPaCa-2 (human pancreatic tumor) were obtained from ATCC and were cultured at 37° C./5% CO₂ in RPMI medium (Cellgro) with 10% fetal bovine serum (FBS), 50 U/ml penicillin G sodium, and 50 U/ml streptomycin sulfate (Gibco/BRL). Cells were treated with TMPyP4 and telomestatin (for the latter molecule, see, e.g., Shin-ya et al., J. Am. Chem. Soc. 123:1262 (2001)) when they reached ˜50% confluency. MiaPaCa cells were first washed with 5 ml of sterile phosphate-buffered saline (PBS, Fisher Scientific). The PBS was aspirated from the cells by vacuum, and replaced with 5 ml of Opti-MEM medium (Gibco-BRL) containing 6 μl Lipofectin (Invitrogen). Compounds then were added to the medium. Cells were allowed to grow at 37° C./5% CO₂ for 6 hours to allow for transfection, and the medium was replaced with normal growth medium. After 24 hours, cells were harvested by adding sufficient trypsin (Gibco/BRL) to cover the cells, and cells were incubated at room temperature for approximately 3 minutes or until cells detached from the flask with firm tapping. The trypsin was neutralized with an equal volume of culture medium, the cells were then pelleted from this mixture by centrifugation at 500×gravity, the supernatant was aspirated, and the pellet was washed in PBS, recentrifuged, and frozen at −80° C.

RET RNA then was extracted from the cells. Cell pellets were thawed and resuspended in 400 μl of a buffer from a commercially available kit (i.e., Buffer RA1 from the NucleoSpin RNA II Kit (Clontech)). Total cellular RNA extraction was performed according to the manufacturer's protocol. Reverse transcription was performed to convert mRNA (i.e., polyadenylated RNA) to cDNA, and RET-specific primers were used in PCR to amplify RET message.

It was demonstrated using MiaPaCa cells that telomestatin at the IC₇₀ concentration downregulated RET RNA expression by at least 50% after 48 hours and 72 hours. TMPyP4 at 100 μM did not significantly downregulate RET but did downregulate CMYC by about 50%. This is in accord with the relative effects in stabilization of the RET chair quadruplex structure, where telomestatin was much more effective than TMPyP4.

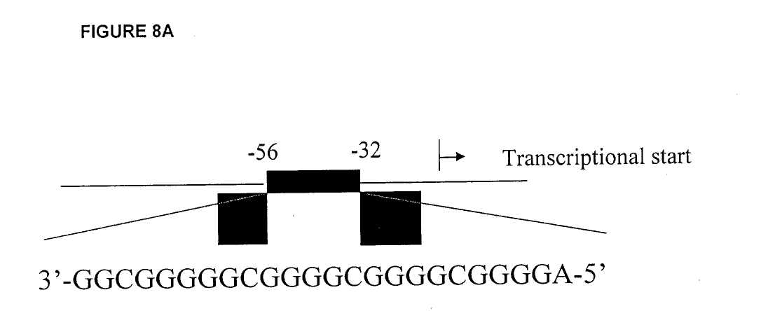

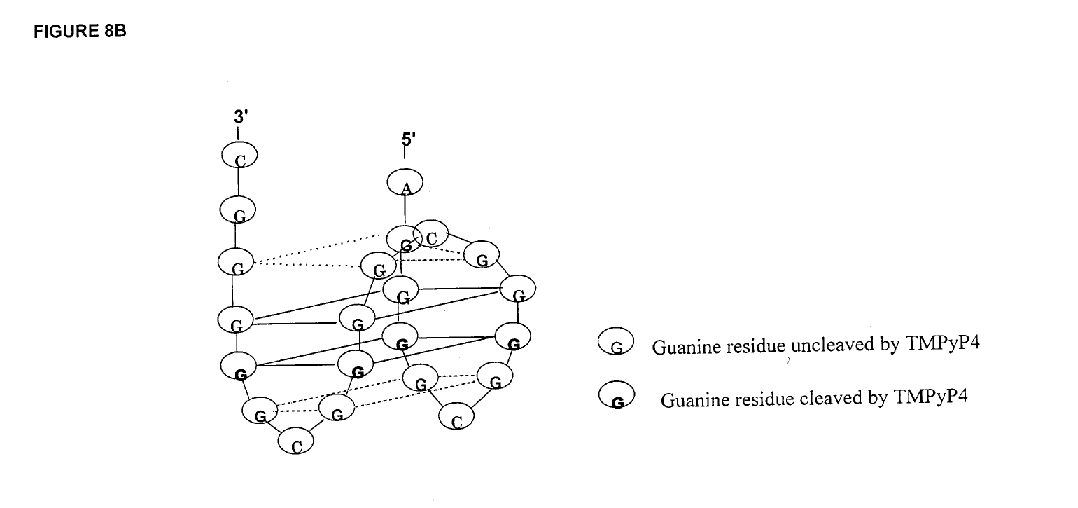

Photo-induced cleavage of the RET quadruplex by TMPyP4 demonstrates that TMPyP4 binds above and below the two stable tetrads shown in black. TMPyP4 cleavage sites are shown in FIG. 8B.

### Example 5

### Identification of Quadruplex-Destabilizing Alleles Associated With Cancer

Normal tissue and colorectal tumor specimens were collected from patients having tumors and/or polyps at the University of Arizona Cancer Center. Tissues were embedded in paraffin and stored in a tissue bank. Paraffin blocks then were microtomed (i.e., thin slices were cut from each) and mounted on glass slides. Six slides were generated from each paraffin block, where two were used for orientation to determine where tumor and normal tissues were located. These slides were stained with hematoxylin and eosin. The other four were utilized for laser capture micro-dissection (LCM) and sequencing. LCM was utilized to collect cells from each tissue specimen. A PixCelII laser capture system (Arcturus) was utilized with the following settings: 30 μm, 50 mW power and 6.2 ms duration. Approximately 1500 pulses were taken and adhered to a CapSure HS LCM cap (Arcturus). A Pico Pure DNA extraction kit was used (Arcturus) to extract genomic DNA from the laser captured cells.

Extracted cells from primary tumor specimens were incubated in 10 μl of proteinase K solution for at least 16 hours at 65° C. The genomic DNA was used in subsequent PCR reactions with NHEseqfw (GACAAGGATGCGGTTTGTCA, SEQ ID NO:) and NHEseqrv (GAGATTAGCGAGAGAGGATC, SEQ ID NO:) primers. Each 5 μl reaction contained 1× high-fidelity PCR buffer (Invitrogen), 50 μM each of dCTP, dATP, dGTP, and dTTP (Fermentas), 2 mM MgSO₄ (Invitrogen), 2.5 U platinum Taq high-fidelity polymerase (Invitrogen), 0.5 μM of each primer, distilled deionized water, and 224 μl of the genomic DNA from above. The reactions were incubated in a DNA Engine Peltier Thermal Cycler as follows: 95° C., 5 minutes; (95° C., 1 minute; 59° C., 1 minute, 10 seconds; 72° C., 1 minute 30 seconds)×45; and then 72° C., 5 minutes. PCR products were held for a time at 4° C. and stored at −20° C. PCR products were resuspended in 100 μL of nuclease-free water and were sequenced using the NHEseqrv primer and an ABI 377 automated sequencer (Applied Biosystems).

The following table depicts sequences of six out of 21 primary tumor samples analyzed in the CMYC NHE quadruplex:

| | | Tumor |
|---|---|---|
| SAMPLE | Sequence | Grade |
| 1 | TGGGGANGGTGAGGAGGGNGGGG | T2N0 |
| 2 | TGGGGAGGGTGGGGANAGTGGGG | T3N0 |
| 3 | TGGGGAGGGTGGGGAGNGTGGGG | T3N0 |
| 4 | TGGGGANGGTGGGGANAGTGGGG | T3N0 |
| 5 | TGGGGAGGGTGGGGAGAGTGGGG | T3N2 |
| 6 | TGGGGAGGGTGAGGAGGGTGGGG | T3N2 |

In the table, the embolded A in samples 1, 2, 4, 5 and 6 represent a guanine to adenine mutation. The embolded N in samples 1-4 depicts a guanine or adenine. The mutations identified in the primary colorectal tumor specimens are at identical positions as the mutations generated in vitro for determining whether the chair or basket quadruplex information was biologically relevant (see Example 2), and are expected to destabilize chair quadruplex structure.

Allelic variants identified in the CMYC-associated quadruplex forming DNA sequence are compared among polyp samples and primary tumor samples. The presence of the allelic variants described above are useful for determining whether a subject is at risk of developing or having colorectal cancer.

### Example 6

### Colorectal Cancer Prognostic and Diagnostic Assay

A colorectal cancer prognostic or diagnostic assay is carried out by obtaining a DNA sample from a subject, determining the nucleotide sequence of a CMYC-associated quadruplex-forming sequence, and identifying the subject as being at risk of developing or having colorectal cancer when the nucleotide sequence corresponds to a quadruplex-destabilizing allele. A colorectal tissue sample is obtained for a subject and then DNA is extracted from the sample. The isolated DNA is quantified and then contacted with PCR primers NHEseqfw (GACAAGGATGCGGTTTGTCA, SEQ ID NO:) and NHEseqrv (GAGATTAGCGAGAGAGGATC, SEQ ID NO:). Each 5 μl reaction contains 1× high-fidelity PCR buffer (Invitrogen), 50 μM each of dCTP, dATP, dGTP, and dTTP (Fermentas), 2 mM MgSO₄ (Invitrogen), 2.5 U platinum Taq high-fidelity polymerase (Invitrogen), 0.5 μM of each primer, distilled/deionized water, and 224 μl of the genomic DNA from above. The reactions are incubated in a DNA Engine Peltier Thermal Cycler as follows: 95° C., 5 minutes; (95° C., 1 minute; 59° C., 1 minute, 10 seconds; 72° C., 1 minute 30 seconds)×45; and then 72° C., 5 minutes. PCR products are held for a time at 4° C. and optionally stored at −20° C. PCR products are resuspended in 100 μL of nuclease-free water and sequenced using the NHEseqrv primer and an ABI 377 automated sequencer. The nucleotide sequence is reviewed and the presence or absence of the following sequences is determined: TGGGGAGGGTGXGGAGGGTGGGG, TGGGGAGGGTGGGGAGXGTGGGG, or TGGGGAGGGTGXGGAGXGTGGGG, where G is guanine, A is adenine, C is cytosine, and T is thymine, and X is a nucleotide other than guanine. Where the tested sequence differs from the preceding sequences and the X nucleotides are not guanine, the DNA extraction and sequencing procedures often are repeated. If one of the above-listed sequences is identified, the subject is prognosed or diagnosed with colorectal cancer. The subject is identified as being at risk of developing or having colorectal cancer when a quadruplex-destabilizing allelic variant is present in polyp samples or in tumor samples.

### Example 7

### Colorectal Cancer Therapeutic

Performing the prognostic or diagnostic procedure described in Example 6, the presence of a cancer-associated allele can be detected. Where the allele has the sequence TGGGGAGGGTGXGGAGGGTGGGG, TGGGGAGGGTGGGGAGXGTGGGG, or TGGGGAGGGTGXGGAGXGTGGGG (G is guanine, A is adenine, C is cytosine, T is thymine, and X is any nucleotide except for guanine), a PNA molecule having the sequence CCCCACCCTCCYCACCCTCCCCA, CCCCACYCTCCCCACCCTCCCCA, or CCCCACYCTCCYCACCCTCCCCA, (where G is guanine, A is adenine, C is cytosine, T is thymine, and Y is a nucleotide complementary to X), respectively, is selected and utilized as a therapeutic. A PNA molecule 20 nucleotides in length or 15 amino acids in length and having a subsequence of the above nucleotide sequences also is utilized as a therapeutic.

PNAs are synthesized using an Applied Biosystems (Foster City, Calif.) Expedite 8909 Synthesizer using monomer Fmoc reagents from Applied Biosystems (Mayfield & Corey, Biorg. Med. Chem Lett. 9:1419-1422 (1999)). PNAs are purified by reverse-phase HPLC and analyzed by time-of-flight mass spectrometry (MALDI-TOF) as described in Mayfield & Corey, supra. PNA is quantified based on spectrophotometric A₂₆₀ values and the conversion factor of 30 μg/ml OD₂₆₀. The PNA molecule often is synthesized with a lysine moiety at the C-terminus.

PNA-peptide conjugates are optionally synthesized. PNA-peptide conjugates are synthesized with either the peptide or the PNA in the C-terminal position. Depending on the orientation, either the peptide is synthesized first by automated synthesis or the PNA is synthesized first by manual synthesis. After completion of this initial synthesis, a small aliquot is deprotected and cleaved, then characterized by MALDI-TOF spectrometry to ensure successful synthesis of the entire lot. Once the identity of the synthesis is confirmed, fully protected oligomer is used as the basis for addition of the PNA by manual synthesis or a peptide by automated synthesis. Boc-protected monomers normally are employed for PNA synthesis. When the PNA is added to the N-terminus of a peptide already prepared by Fmoc synthesis, Fmoc chemistry also is used for the PNA synthesis. PNA-peptide conjugates are purified using the procedure described above or by using a Rainin HPLC system with a Dynamax detector set at 260 nm using a Delta Pak C18 300 Å column (7.8×300 mm) heated to 50° C. (see e.g. Wang et al., J. Mol. Biol. 313:933-940 (2001)). The peptide conjugated to PNA is an Antennapedia homeodomain peptide (i.e. GGRQIWFQNRMKWKK, GGLWFQNRMKWKKEN, GGGRQIKIWFQNRRMKWKK, or GGGKIWFQNRRMKWKKEN) or an HIV tat peptide (i.e. SEQ ID NO: 2-7 in U.S. Pat. No. 5,652,122). Where the N-terminus of the PNA is linked to the C-terminus of the peptide, the C-terminus of the PNA ends with a lysine moiety.

Fluorescent-labeled PNAs and PNA-peptide conjugates are optionally synthesized. Fluorescent-labeled PNAs and PNA-peptide conjugates are useful for detecting cells transfected with the PNA or PNA-peptide conjugate and for sorting transfected cells. PNA-peptide conjugates typically are labeled with fluorescein or rhodamine. Fluorescein maleimide is coupled to deprotected PNA-peptide conjugates through cysteine. Rhodamine can withstand trifluormethanesulfonic acid (TFMSA) cleavage conditions as well as four hours of TFA cleavage without breaking down and is added to the N-terminus of the fully protected PNA or PNA-peptide hybrid before cleavage. After the N-terminal Boc or Fmoc protecting group is removed from the completed PNA-peptide hybrid, rhodamine is coupled using diisopropylethtlamine (DIPEA) to increase the pH to 9.0. Coupling is complete after thirty minutes. At least a four-fold excess of rhodamine over the PNA-peptide is used, while fluorescein is used in twofold excess. After coupling, the finished product is washed extensively with DMF or NMP to remove the unreacted rhodamine.

The PNA or PNA-peptide conjugate then is transfected into cells in vitro or is delivered by intravenous administration to a subject. In either application, the PNA and PNA-peptide conjugates are formulated before delivery. In one application, PNA formulations are prepared by equilibrating 15 μl of 100 μM PNA in 135 μl of Opti-MEM (Life Technologies). In a separate tube, 4.5 μl of (7 μg/ml) LipofectAMINE (Life Technologies) is activated in 145.5 μl of Opti-MEM by vigorously shaking for 5 s followed by equilibration for 5-10 min at room temperature. LipofectAMINE is obtained from Life Technologies (Gaithersburg, Md.) and solubilized according to the manufacturer protocol in sterile water. The PNA and LipofectAMINE aliquots (300 μL each) are mixed together and agitated vigorously for 15 s. Lipid complexes are allowed to form by incubating the mixture at room temperature for 15-20 min in the dark. The solution containing the PNA-lipid complex (600 μl) is diluted to 3 ml with Opti-MEM to afford a solution containing 1 μM PNA. This solution then is diluted to a final working concentration, which is 100 nM in most cases.

For transfection in vitro, cells are plated at 11000-13000 cells/well in 48-well plates using Dulbecco's MEM (minimal essential media) with glutamine supplemented with 10% superstripped fetal calf serum, 20 mM HEPES buffer (final concentration, pH 7.4), 500 units/ml penicillin, 0.1 mg/ml streptomycin, and 0.06 mg/ml anti-PPLO reagent (Life Technologies). Superstripped serum is used to ensure that competing ligands are removed from serum prior to addition of molecules. Ligand stripping is achieved by twice extracting serum with activated charcoal and cation exchange (CAG 1-X8 resin, Bio-Rad, Hercules, Calif.). Superstripped serum is doubly filtered through a 0.2 μM filter prior to addition to media. Cells are incubated at 37° C. at 5% CO₂ for a minimum of 6 h prior to initiating transfection. The cells then are washed once with 250 μL of Opti-MEM, followed by overnight transfection with PNA-lipid complex.

In an alternative in vitro transfection procedure that does not utilize lipid-formulated PNA or PNA-peptide conjugates, cells are allowed to attach to 24-well plates in IX Dulbecco's Modified Eagle's Media (DMEM) (Mediatech, Herndon Va.) supplemented with 10% fetal bovine serum. Media is removed from cells and PNAs and conjugates are added directly for three minutes prior to addition of fresh media to bring the final concentration of oligomer to 1 μM. Cells are incubated for one to twelve hours, with maximal uptake observed after one hour. Following incubation, cells are rinsed 8-12 times with phosphate buffered saline (PBS) to remove residual free fluorescent material when fluorescent-tagged molecules are utilized. Cells then are treated with trypsin and transferred to Lab-TekII chamber slides (Nalge-Nunc, Rochester, N.Y.) for visualization. After reattachment, cells are washed several times with PBS and fixed with 70% methanol. Vectashield (Vector Laboratories, Burlingame Calif.) mounting medium (25 μL) is added to the fixed slides. Cells are visualized using an Olympus BHS microscope with a reflected light fluorescence attachment.

Transfected cells optionally are analyzed by flow cytometry. Adherent populations of cells are treated with 1 μM rhodamine labeled PNA or PNA-conjugate for 2 h at 37° C. Cells are extensively washed, trypsinized, and resuspended in 0.5 ml 1×PBS. Populations are immediately analyzed on a FACStarPlus flow cytometer using LYSYS II software (Becton Dickinson, Franklin Lakes, N.J.) and a 575 nm broad band pass filter. Cell populations are gated to measure only fluorescence in intact cells.

### Example 8

### Quadruplex Assays

Test molecules identified as quadruplex interacting molecules and quadruplex-forming nucleic acids often are further confirmed for quadruplex-forming activity or quadruplex-interacting activity in assays described hereafter. These assays include mobility shift assays, DMS methylation protection assays, polymerase arrest assays, transcription reporter assays, circular dichroism assays, and fluorescence assays.

### Gel Electrophoretic Mobility Shift Assay (EMSA)

An EMSA is useful for determining whether a nucleic acid forms a quadruplex and whether a nucleotide sequence is quadruplex-destabilizing. EMSA is conducted as described previously (Jin & Pike, Mol. Endocrinol. 10: 196-205 (1996)) with minor modifications. Synthetic single-stranded oligonucleotides are labeled in the 5′-terminus with T4-kinase in the presence of [α-³²P] ATP (1,000 mCi/mmol, Amersham Life Science) and purified through a sephadex column. ³²P-labeled oligonucleotides (˜30,000 cpm) then are incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25× Tris borate-EDTA buffer (0.25×TBE, 1×TBE is 89 mM Tris-borate, pH 8.0, 1 mM EDTA). The gel is dried and each band is quantified using a phosphorimager.

### DMS Methylation Protection Assay

Chemical footprinting assays are useful for assessing quadruplex structure. Quadruplex structure is assessed by determining which nucleotides in a nucleic acid are protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoathanol:water:NaOAc (3 M); 1:6:7; v:v:v). Following ethanol precipitation (twice) and piperidine cleavage, the reactions are separated on a preparative gel (16%) and visualized on a phosphorimager.

### Polymerase Arrest Assay

An example of the Taq polymerase stop assay is described in Han et al., Nucl. Acids Res. 27: 537-542 (1999), which is a modification of that used by Weitzmann et al., J. Biol. Chem. 271, 20958-20964 (1996). Briefly, a reaction mixture of template DNA (50 nM), Tris.HCl (50 mM), MgCl₂ (10 mM), DTT (0.5 mM), EDTA (0.1 mM), BSA (60 ng), and 5′-end-labeled quadruplex nucleic acid (˜18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl), and 10 mg bromophenol blue), the reactions are separated on a preparative gel (12%) and visualized on a phosphorimager. Adenine sequencing (indicated by “A” at the top of the gel) is performed using double-stranded DNA Cycle Sequencing System from Life Technologies. The general sequence for the template strands is TCCAACTATGTATAC-INSERT-TTAGCGACACGCAATTGCTATAGTGAGTCGTATTA. Bands on the gel that exhibit slower mobility are indicative of quadruplex formation.

### Transcription Reporter Assay

A luciferase promoter assay described in He et al., Science 281: 1509-1512 (1998) often is utilized for the study of quadruplex formation. Specifically, a vector utilized for the assay is set forth in reference 11 of the He et al. document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

### Circular Dichroism Assay

Circular dichroism (CD) is utilized to determine whether another molecule interacts with a quadruplex nucleic acid. CD is particularly useful for determining whether a PNA or PNA-peptide conjugate hybridizes with a quadruplex nucleic acid in vitro. PNA probes are added to quadruplex DNA (5 μM each) in a buffer containing 10 mM potassium phosphate (pH 7.2) and 10 or 250 mM KCl at 37° C. and then allowed to stand for 5 min at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for quadruplex DNA alone, PNA alone, and quadruplex DNA with PNA are generated to determine the presence or absence of an interaction (see, e.g., Datta et al., JACS 123:9612-9619 (2001)). Spectra are arranged to represent the average of eight scans recorded at 100 nm/min.

### Fluorescence Binding Assay

50 μl of quadruplex nucleic acid or a nucleic acid not capable of forming a quadruplex is added in 96-well plate. A test molecule or quadruplex-targeted nucleic acid also is added in varying concentrations. A typical assay is carried out in 100 μl of 20 mM HEPES buffer, pH 7.0, 140 mM NaCl, and 100 mM KCl. 50 μl of the signal molecule N-methylmesoporphyrin IX (NMM) then is added for a final concentration of 3 μM. NMM is obtained from Frontier Scientific Inc, Logan, Utah. Fluorescence is measured at an excitation wavelength of 420 nm and an emission wavelength of 660 nm using a FluroStar 2000 fluorometer (BMG Labtechnologies, Durham, N.C.). Fluorescence often is plotted as a function of concentration of the test molecule or quadruplex-targeted nucleic acid and maximum fluorescent signals for NMM are assessed in the absence of these molecules.

The contents of each document cited herein is incorporated by reference in its entirety.

What is claimed is:

1. A method for identifying a compound that modulates the biological activity of a native quadruplex DNA, which comprises
contacting a test quadruplex DNA in a chair conformation with a candidate compound; and
determining the presence or absence of an interaction between the candidate compound and the test quadruplex DNA, whereby the candidate compound that interacts with the test quadruplex DNA is identified as the compound that modulates the biological activity of the native quadruplex DNA.

2. The method of claim 1, wherein the test quadruplex DNA comprises a nucleotide sequence selected from the group consisting of (GₐX_{b})_{c}Gₐ, wherein
G is guanine;
X is guanine, cytosine, adenine, or thymine;
a is an integer between 2 to 6;
b is an integer between 1 to 4; and
c is the integer 3.

3. The method of claim 1, wherein the native quadruplex DNA comprises a nucleotide sequence selected from the group consisting of
TG₄AG₃TG₄AG₃TG₄AAGG;
CTAGAG₅CG₅CG₅CG₅AG₄T;
G₈ACGCG₃AGCTG₅AG₃CTTG₄CCAG₃CG₄CGCTTAG₅;
AGGAAG₄AG₃CCG₆AGGTGGC;
G₅(CG₄)₃;
G₃AGGAAG₅CG₃AGTCG₄;
G₄ACGCG₃CG₅CG₆AG₃CG;
C₃G₄CG₃C₂G₅CG₄TC₃G₂C4₅CG₂AG;
(G₃A)₃AGGA(G₃A)₄GC; and
G₅(CG₄)₃.

4. The method of claim 1, wherein the native quadruplex DNA comprises a nucleotide sequence that is identical to a nucleotide sequence in the test quadruplex DNA.

5. The method of claim 1, wherein the chair conformation of the test quadruplex DNA is formed by incubating the DNA in a solution comprising potassium ions for a time period less than the time period required to form the basket conformation of the quadruplex DNA.

6. The method of claim 1, wherein the test quadruplex DNA comprises a mutation that prevents formation of the basket conformation.

7. The method of claim 1, wherein the test quadruplex DNA is coupled to a reporter expression system.

8. The method of claim 7, wherein the reporter expression system comprises a luciferase reporter.

9. The method of claim 1, wherein the interaction is assayed by a Taq polymerase arrest assay.

10. A method for identifying a compound that binds a native quadruplex DNA, which comprises
contacting a test quadruplex DNA in a chair conformation with a candidate compound; and
determining the presence or absence of binding between the candidate compound and the test quadruplex DNA, whereby the candidate compound that binds the test quadruplex DNA is identified as the compound that binds the native quadruplex DNA.

11. The method of claim 10, wherein the test quadruplex DNA comprises a nucleotide sequence selected from the group consisting of (GₐX_{b})_{c}Gₐ, wherein
G is guanine;
X is guanine, cytosine, adenine, or thymine;
a is an integer between 2 to 6;
b is an integer between 1 to 4; and
c is the integer 3.

12. The method of claim 10, wherein the native quadruplex DNA comprises a nucleotide sequence selected from the group consisting of
TG₄AG₃TG₄AG₃TG₄AAGG;
CTAGAG₅CG₅CG₅CG₅AG₄T;
G₈ACGCG₃AGCTG₅AG₃CTTG₄CCAG₃CG₄CGCTTAG₅;
AGGAAG₄AG₃CCG₆AGGTGGC;
G₅(CG₄)₃;
G₃AGGAAG₅CG₃AGTCG₄;
G₄ACGCG₃CG₅CG₆AG₃CG;
C₃G₄CG₃C₂G₅CG₄TC₃G₂C4₅CG₂AG;
(G₃A)₃AGGA(G₃A)₄GC; and
G₅(CG₄)₃.

13. The method of claim 10, wherein the native quadruplex DNA comprises a nucleotide sequence that is identical to a nucleotide sequence in the test quadruplex DNA.

14. The method of claim 10, wherein the chair conformation of the quadruplex DNA is forned by incubating the DNA in a solution comprising potassium ions for a time period less than the time period required to form the basket conformation of the quadruplex DNA.

15. The method of claim 10, wherein the quadruplex DNA comprises a mutation that prevents formation of the basket conformation.

16. A method for modulating the biological activity of a biologically significant native quadruplex DNA, which comprises contacting a system comprising the native quadruplex DNA with a compound which interacts with a quadruplex DNA in a chair conformation; whereby the compound modulates the biological activity of the native quadruplex DNA.

17. The method of claim 16, wherein the native quadruplex DNA comprises a nucleotide sequence selected from the group consisting of (GₐX_{b})_{c}Gₐ, wherein
G is guanine;
X is guanine, cytosine, adenine, or thymine;
a is an integer between 2 to 6;
b is an integer between 1 to 4; and
c is the integer 3.

18. The method of claim 16, wherein the native quadruplex DNA comprises a nucleotide sequence selected from the group consisting of
TG₄AG₃TG₄AG₃TG₄AAGG;
CTAGAG₅CG₅CG₅CG₅AG₄T;
G₈ACGCG₃AGCTG₅AG₃CTTG₄CCAG₃CG₄CGCTTAG₅;
AGGAAG₄AG₃CCG₆AGGTGGC;
G₅(CG₄)₃;
G₃AGGAAG₅CG₃AGTCG₄;
G₄ACGCG₃CG₅CG₆AG₃CG;
C₃G₄CG₃C₂G₅CG₄TC₃G₂C4₅CG₂AG;
(G₃A)₃AGGA(G₃A)₄GC; and
G₅(CG₄)₃.

19. The method of claim 16, wherein the system is a cell.

20. The method of claim 16, wherein the system is an organism.

21. An isolated nucleic acid which comprises a nucleotide sequence selected from the group consisting of
TG₄AG₃TG₄AG₃TG₄AAGG;
CTAGAG₅CG₅CG₅CG₅AG₄T;
G₈ACGCG₃AGCTG₅AG₃CTTG₄CCAG₃CG₄CGCTTAG₅;
AGGAAG₄AG₃CCG₆AGGTGGC;
G₅(CG₄)₃;
G₃AGGAAG₅CG₃AGTCG₄;
G₄ACGCG₃CG₅CG₆AG₃CG;
C₃G₄CG₃C₂G₅CG₄TC₃G₂C4₅CG₂AG;
(G₃A)₃AGGA(G₃A)₄GC; and
G₅(CG₄)₃;
wherein one or more guanines is substituted with another nucleotide which destabilize the chair quadruplex conformation of the isolated nucleic acid.

22. The isolated nucleic acid of claim 21, wherein the nucleotide sequence is selected from the group consisting of TGGGGAGGGTGXGGAGGGTGGGG, TGGGGAGGGTGGGGAGXGTGGGG, or TGGGGAGGGTGXGGAGXGTGGGG, wherein G is guanine, A is adenine, C is cytosine, T is thymine, and X is a nucleotide other than guanine.

23. A method for determining whether a subject is at risk of developing cancer or at risk of having cancer, which comprises detecting the presence or absence of a polymorphic variant in a nucleic acid sample from the subject, wherein the polymorphic variation destabilizes a native quadruplex structure; whereby the presence of the polymorphic variant determines that the subject is at risk of developing the cancer or having the cancer.

24. The method of claim 23, wherein the cancer is colorectal cancer.

25. The method of claim 24, wherein the polymorphic variant is TGGGGAGGGTGXGGAGGGTGGGG, TGGGGAGGGTGGGGAGXGTGGGG, or TGGGGAGGGTGXGGAGXGTGGGG, wherein G is guanine, A is adenine, C is cytosine, T is thymine, and X is a nucleotide other than guanine.

26. The method of claim 25, wherein X is adenine.

27. The method of claim 23, wherein the nucleic acid sample is isolated from a colorectal tissue sample.

28. A method for detecting the presence or absence of cancer in a subject, which comprises detecting the presence or absence of a polymorphic variant in a nucleic acid sample from the subject, wherein the polymorphic variation destabilizes a native quadruplex structure; and if the presence of the polymorphic variant is detected in the nucleic acid sample, performing a further examination of the subject to detect the presence or absence of cancer.

29. The method of claim 28, wherein the cancer is colorectal cancer and the further examination is a colorectal examination.

30. The method of claim 29, which further comprises removing one or more polyps if polyps are identified in the colorectal examination.

31. A method for treating cancer in a subject, which comprises detecting the presence or absence of a polymorphic variant in a nucleic acid sample from the subject, wherein the polymorphic variation destabilizes a native quadruplex structure; and if the presence of the polymorphic variant is detected in the nucleic acid sample, treating the subject with a cancer treatment.

32. The method of claim 31, wherein the cancer is colorectal cancer and the cancer treatment is selected from the group consisting of surgical removal of the colorectal cancer, chemotherapy, radiation therapy, or a combination thereof.

33. The method of claim 31, wherein the cancer is colorectal cancer and the anticancer treatment is administration of a nucleic acid that specifically hybridizes to the polymorphic variant.

34. The method of claim 33, wherein the nucleic acid administered is a peptide nucleic acid.

35. The method of claim 34, wherein polymorphic variant comprises the nucleotide sequence TGGGGAGGGTGXGGAGGGTGGGG, TGGGGAGGGTGGGGAGXGTGGGG, or TGGGGAGGGTGXGGAGXGTGGGG, and the peptide nucleic acid comprises all or a portion of the nucleotide sequence CCCCACCCTCCYCACCCTCCCCA, CCCCACYCTCCCCACCCTCCCCA, or CCCCACYCTCCYCACCCTCCCCA, wherein G is guanine, A is adenine, C is cytosine, T is thymine, X is a nucleotide other than guanine, and Y is a nucleotide complementary to X.

36. The method of claim 33, wherein the nucleic acid is administered by colorectal delivery.
